(19) United States
(12) Patent Application Publication
(10) Pub. No.: US 2023/0312721 A1
(43) Pub. Date: Oct. 5, 2023

(54) Anti-B7H3 Antibodies for the Treatment of Cancer

(71) Applicant: Y-mAbs Therapeutics, Inc., New York, NY (US)

(72) Inventors: Ahmed MAHIUDDIN, Verona, NJ (US); Sonia SEQUEIRA, Verona, NJ (US); Linlin WANG, Mountain Lakes, NJ (US)

(21) Appl. No.: 17/924,161

(22) PCT Filed: Jun. 1, 2021
(86) PCT No.: PCT/DK2021/050170

§ 371 Date: Nov. 9, 2022

Related U.S. Application Data
(60) Provisional application No. 63/034,592, filed on Jun. 4, 2020.

(51) Int. Cl. C07K 16/28; A61K 51/10; C07K 16/44; A61P 35/00

(52) U.S. Cl. CPC C07K 16/2827; A61K 51/1027; C07K 16/44; A61P 35/00; A61K 2039/505

(57) ABSTRACT

The present invention relates to humanized antibodies and antigen binding fragments. More specifically, the invention relates to humanized antibodies and antigen binding fragment, capable of binding to antigens, wherein said antibodies or antigen binding fragments comprises at least 50% amino acids that are identical to the amino acids of the human germline of said antibody or antigen binding fragment. The present invention further relates to antibodies with minimal potential for immunogenicity by intraperitoneal or systemic administration to treat B7-H3(+) solid tumors. The invention further relates to fully humanized antibodies against B7-H3 with high human content, strong binding to B7-H3, high stability, high purity, and high expression titers.

### FIELD OF THE INVENTION

The present invention relates to humanized antibodies and antigen binding fragments. More specifically, the invention relates to humanized antibodies and antigen binding fragments, capable of binding to antigens, wherein said antibodies or antigen binding fragments comprises preferably at least 50%, more preferred at least 75%, amino acids that are identical to the amino acids of the human germline of said antibody or antigen binding fragment. The present invention further relates to antibodies with minimal potential for immunogenicity by intraperitoneal or systemic administration to treat B7-H3(+) solid tumors. The invention further relates to fully humanized antibodies against B7-H3 with high human content, strong binding to B7-H3, high stability, high purity, and high expression titers.

### BACKGROUND

Human B7-H3 (also named as CD276) is a member of the B7/CD28 immunoglobulin superfamily. According to Chapoval et al, B7-H3 was identified as a type I transmembrane protein, containing two isoforms, 2Ig B7H3 with a single extracellular V- and C-like Ig domains[1] and, according to Steinberger et al, 4Ig B7H3 with a duplicate of V- and C-like Ig domains in tandem[2]. According to Steinberger et al, B7-H3 was initially found to be a co-stimulatory molecule that can induce IFN-γ. B7-H3 was later proven to inhibit T cell proliferation and is correlated with decrease in production of IFN-γ[2].

According to International patent application WO2016033225, humanized and/or affinity matured versions of an 8H9 antibody are described. According to WO2016033225 four humanized 8H9 (hu8H9) IgG1s have been made. The variant which contained the most human content, did not bind to B7-H3 as strongly as the three other variants, although none displayed optimal binding capacity. Six mutations were introduced into one of the hu8H9 antibodies to create hu8H9 H3L3, and an additional 12 humanizing mutations were incorporated into hu8H9 H3L3 to generate hu8H9 4.1. Six affinity maturation mutations were incorporated into the hu8H9 H3L3 sequence to generate hu8H9 3.1 scFv and IgG1 variants. Five affinity maturation mutations were incorporated into the hu8H9 H3L3 to generate hu8H9 5.1 scFv and IgG1 variants.

### SUMMARY OF THE INVENTION

According to Loos et al and Modak et al, B7-H3 is widely expressed on a variety of human solid tumors, including pediatric solid tumors, such as brain tumors and sarcomas[3, 4]. However, the expression in normal human tissues is limited. Moreover, B7-H3 expression on solid tumor was found to be correlated with poor patient survival, increased risk of clinical cancer recurrence, cancer-specific death and more in multiple cancers, including prostate cancer, pancreatic cancer, gastric cancer, ovarian cancer, osteosarcoma, neuroblastoma and glioblastoma, etc.[5-11]. B7-H3 is an ideal target for immunotherapy[12-14]. Anti-B7-H3 mouse monoclonal antibody 8H9 has been successfully used as a radioimmunotherapy by compartmental intrathecal delivery to treat patients with recurrent metastatic central nervous system neuroblastoma[15] and by single intraperitoneal administration to patients with desmoplastic small round cell tumors and other solid tumors involving the peritoneum in clinical trials.

As one of the leading causes of cancer-related death worldwide, metastatic gastric cancer has poor outcomes with a reported median survival of one year[16]. The 5-year survival rate of women with metastatic epithelial ovarian cancer remains at 30% [Siegel et al. 2020, American Cancer Society, 2020]. To minimize the potential for immunogenicity of anti-B7-H3 antibodies by intraperitoneal or systemic administration to treat these and other B7-H3(+) solid tumors, here we present fully humanized antibodies, Hu8H9 antibody against B7-H3 with high human content, strong binding to B7-H3, high stability, high purity, and high expression titers. A previous attempt to humanize murine mAb 8H9 had low human germline content in the variable domains (73% human germline content in the V-Kappa domain and 76.5% human germline content in the VH domain) and required three affinity maturation mutations in the CDR region to recover the affinity of murine 8H9 [17].

For B7-H3 targeted delivery of radioactive payload and to eliminate antibody interaction with effector cells or complement, we made the mutations “N298A and K323A” and “L235A, L236A and K323A” on the Fc of IgGs (N297A and K322A; L234A, L235A and K322A as numbered according to Kabat) to eliminate ADCC (antibody-dependent cellular cytotoxicity), ADCP (antibody-dependent cellular phagocytosis), and complement activation. The modified Fc is silent in effect function, i.e. null Fc.

Based on the sequences of leading candidates, we also describe a novel class of SADA (Self-Assembly and DisAssembly) antibodies that bind to both human B7-H3 and DOTA (Dodecane Tetraacetic Acid, 1,4,7,10-Tetraazacyclododecane-1,4,7,10-tetraacetic acid, chelator for metal) for pre-targeted radioimmunotherapy. The SADA antibody contains a ScFv against B7-H3 and a ScFv against DOTA (based on humanized C825 antibody from Patent WO2016130539A2, all of which is incorporated by reference in its entirety). This type of construct can tetramerize and monomerize in vivo for faster clearance without using clearing agents.

In certain embodiments, the antibody or antigen binding fragment thereof is linked to a self-assembly disassembly (SADA) polypeptide disclosed in International Patent Application Publication No. WO2018204873, all of which is incorporated by reference in its entirety.

In certain embodiments the tetramerization domain is identical to SEQ ID NO. 139.

In certain embodiments, the antibody or antigen binding fragment thereof comprises an engineered protein with high affinity for DOTA chelates, disclosed in U.S. Pat. No. 8,648,176 or International Patent Application Publication No. WO2010099536 all of which is incorporated by reference in its entirety.

According to an aspect the invention concerns a humanized antibody or antigen binding fragment thereof, capable of binding to B7H3 antigen, comprising

CDR regions having a total of at least 90% identity to the CDR sequences selected among SEQ ID No. 25-30 or 64-99, and FR regions having a total of at least 70% identity to the FR sequences selected among SEQ ID No. 40-63 or 108-131.

Preferably at least 75%.

CDR regions refers to the complementarity determining regions (CDR), while FR regions refers to framework regions. A framework region is a subdivision of the variable region (Fab) of the antibody. The variable region is composed of seven amino acid regions, four of which are framework regions and three of which are hypervariable regions. The framework regions are responsible for acting as a scaffold for the complementarity determining regions (CDR), also referred to as hypervariable regions, of the Fab.

It is a problem to humanize a murine antibody in a way so the affinity to the antigen is retained, while introducing a degree of humanization so adverse reactions such as side effects are avoided. It is further a problem to obtain an antibody which exhibits good stability and has potential for being used as a drug in terms of efficacy and safety.

In certain embodiments the stability of antibodies or antibody fragments can be improved using techniques known in the art, such as introducing additional disulfide bonds and substituting oxidation labile residues. Disulfide bonds may be formed between the thiol groups of cysteine residues. Introducing additional disulfide bonds may be done by inspection of the structure of the antibody or antibody fragment, identifying residues in the framework regions within a suitable distance for disulfide bonds, and substituting the amino acids in these positions with cysteine residues that may form the disulfide bonds. Preferred examples of suitable positions for introducing cysteine residues in order to form additional disulfide bonds are position 3 in LFR4 and position 9 in HFR2. Without being bound by theory it is surprising that by changing the LFR regions it is possible to keep the sequences of the CDR regions close or identical to the non-humanized murine antibody, thus retaining the affinity, and still allowing a high degree of humanization of the antibody.

LFR may be defined as a Light chain framework region. HFR may be defined as a heavy chain framework region.

According to another aspect, the invention concerns a humanized antibody or antigen binding fragment thereof, capable of binding to B7H3 antigen, comprising regions having a total at least 90% identity to SEQ ID No. 28-30, 67-69, 73-75, 79-81, 85-87, 91-93, preferably at least 95% identity, e.g. at least 96% identity, e.g. at least 97% identity; e.g. at least 98% identity, e.g. at least 99% identity or preferably 100% identity; regions having the sequence of one of SEQ ID No. 25-27, 64-66, 70-72, 76-78, 82-84, 88-90, 94-99, or regions having a sequence that differs from one of SEQ ID No. 25-27, 64-66, 70-72, 76-78, 82-84, 88-90, 94-99 in 1, 2 or 3 Positions, wherein the differences are selected among substitutions, deletions or insertions;

regions having a total at least 75% identity to the sequences according to SEQ ID No. 40-43, 48-51, 56-59 or 108-119, and

regions having a total at least 75% identity to the sequences according to SEQ ID No. 44-47, 52-55, 60-63 or 120-131.

According to another aspect, the invention concerns a humanized antibody or antigen binding fragment thereof, comprising

regions having a total at least 90%, more preferred 95% identity to SEQ ID No. 28-30, 67-69, 73-75, 79-81, 85-87, 91-93, and/or

regions having a total at least 75% identity to SEQ ID No. 40-43, 48-51, 56-59 or 108-119.

According to another aspect, the invention concerns a humanized antibody or antigen binding fragment thereof, comprising regions having a total at least 80%, more preferred 85, preferably 90%, more preferred 95% identity to SEQ ID No. 40-43, 48-51, 56-59 or 108-119.

According to another aspect, the invention concerns a humanized antibody or antigen binding fragment thereof, comprising

regions having a total at least 90%, more preferred 95% identity to SEQ ID No. 25-27, 64-66, 70-72, 76-78, 82-84, 88-90, 94-99, and/or

regions having a total at least 75% identity to SEQ ID No. 44-47, 52-55, 60-63 or 120-131.

According to another aspect, the invention concerns a humanized antibody or antigen binding fragment thereof, comprising regions having a total at least 80%, more preferred 85, preferably 90%, more preferred 95% identity to SEQ ID No. 44-47, 52-55, 60-63 or 120-131.

According to another aspect, the invention concerns a humanized antibody or antigen binding fragment thereof, comprising regions selected among the SEQ ID No. 25-30 and 64-99.

According to another aspect, the invention concerns a humanized antibody or antigen binding fragment thereof, comprising

regions selected among SEQ ID No. 28-30, 67-69, 73-75, 79-81, 85-87, 91-93,

regions selected among SEQ ID No. 25-27, 64-66, 70-72, 76-78, 82-84, 88-90, 94-99,

regions selected among SEQ ID No. 40-43, 48-51, 56-59 or 108-119, and

regions selected among SEQ ID No. 44-47, 52-55, 60-63, or 120-131.

According to another aspect, the invention concerns a humanized antibody or antigen binding fragment thereof, comprising Fc, Fc2 or Null-Fc.

According to another aspect, the invention concerns a humanized antibody or antigen binding fragment thereof, capable of binding to B7H3 antigen, wherein said antibody or antigen binding fragment comprises sequences having at least 70% identity to sequences selected among any of the sequences SEQ ID No. 36, 37, 38 and 39.

According to another aspect, the invention concerns an antibody or antigen binding fragment thereof, capable of binding to an antigen, wherein said antibody or antigen binding fragment comprises a sequence according to SEQ ID No. 15, 16 17 or 132.

According to another aspect, the invention concerns a self-assembly disassembly (SADA) polypeptide, wherein said polypeptide is linked to an antibody or antigen binding fragment according to the invention.

According to another aspect, the invention concerns a polypeptide conjugate comprising:

a self-assembly disassembly (SADA) polypeptide according to the invention, and

an antibody or antigen binding fragment according to the invention.

According to another aspect, the invention concerns a polypeptide conjugate comprising a self-assembly disassembly (SADA) polypeptide, and wherein said conjugate further comprises the bispecific antibody according to the invention, wherein said first antigen is B7H3 and wherein said second antigen is DOTA.

According to another aspect, the invention concerns a polypeptide conjugate comprising a self-assembly disassembly (SADA) polypeptide, and at least a first binding domain that binds to a first target and is covalently linked to the SADA polypeptide.

According to another aspect, the invention concerns an isolated nucleic acid molecule encoding the antibody or antigen binding fragment according to the invention.

According to another aspect, the invention concerns an isolated nucleic acid molecule comprising a sequence according to SEQ ID No. 18, 19, 20, 21 or 22.

According to another aspect, the invention concerns a recombinant vector comprising the isolated nucleic acid molecule according to the invention.

According to another aspect, the invention concerns a host cell comprising the recombinant vector according to the invention.

According to another aspect, the invention concerns a method for the production of an antibody or antigen binding fragment thereof according to the invention comprising a step of culturing the host cell according to the invention in a culture medium under conditions allowing the expression of the antibody or fragment and separating the antibody or fragment from the culture medium.

According to another aspect, the invention concerns a chimeric antigen receptor (CAR) comprising an antibody or antigen binding fragment according to the invention.

According to another aspect, the invention concerns a CAR-T cell expressing a CAR.

According to another aspect, the invention concerns a population of CAR-T cells.

According to another aspect, the invention concerns a composition comprising the population of CAR-T cells.

According to another aspect, the invention concerns a CAR-NK cell expressing a CAR.

According to another aspect, the invention concerns a population of CAR-NK cells.

According to another aspect, the invention concerns a composition comprising the population of CAR-NK cells.

According to another aspect, the invention concerns a pharmaceutical composition comprising the antibody or antigen binding fragment according to the invention.

According to another aspect, the invention concerns a T cell armed with the antibody or antigen binding fragment according to the invention.

According to another aspect, the invention concerns a method of treating, preventing, alleviating and/or diagnosing the symptoms of a medical condition in a subject, comprising a step of administration of an antibody, an antigen binding fragment, a bispecific antibody, a trispecific antibody, a polypeptide conjugate, a composition and/or a CAR into the peritoneum, and wherein said medical condition is characterized by expression of B7H3 antigen.

According to another aspect, the invention concerns a method of imaging a tumor in the peritoneum, wherein said imaging comprises use of an antibody or antigen binding fragment thereof, wherein said tumor is characterized by expression of B7H3 antigen, and wherein the method of imaging has been preceded by a step of administration of an antibody or antigen binding fragment thereof into the peritoneum.

According to another aspect, the invention concerns use of the composition according to the invention in the manufacturing of a medicament for the treatment of a cancer, for use in a method according to the invention.

According to another aspect, the invention concerns use of the antibody of antigen binding fragment according to the invention in the manufacturing of a medicament for the treatment of a cancer and/or for use in a method according to the invention.

According to another aspect, the invention concerns an in vitro use of an antibody or antigen binding fragment thereof according to the invention.

According to another aspect, the invention concerns a method of treating, preventing, alleviating and/or diagnosing the symptoms of a medical condition in a subject, comprising a step of administration of an antibody or antigen binding fragment according to the invention, into the peritoneum, and wherein said medical condition is characterized by expression of B7H3 antigen.

According to another aspect, the invention concerns a method of imaging a tumor in the peritoneum, wherein said imaging comprises use of an antibody or antigen binding fragment thereof according to the invention, wherein said medical tumor is characterized by expression of B7H3 antigen, and wherein the method of imaging has been preceded by a step of administration of an antibody or antigen binding fragment thereof according to the invention into the peritoneum.

### DETAILED DESCRIPTION

According to an embodiment, the invention concerns a humanized antibody or antigen binding fragment thereof, capable of binding to B7H3 antigen, comprising

CDR regions having a total of at least 90% identity to the CDR sequences selected among SEQ ID No. 25-30 or 64-99, and

FR regions having a total of at least 70% identity to the FR sequences selected among SEQ ID No. 40-63 or 108-131.

Preferably at least 75%.

CDR regions refers to the complementarity determining regions (CDR), while FR regions refers to framework regions. A framework region is a subdivision of the variable region (Fab) of the antibody. The variable region is composed of seven amino acid regions, four of which are framework regions and three of which are hypervariable regions. The framework regions are responsible for acting as a scaffold for the complementarity determining regions (CDR), also referred to as hypervariable regions, of the Fab.

It is a problem to humanize a murine antibody in a way so the affinity to the antigen is retained, while introducing a degree of humanization so adverse reactions such as side effects are avoided. It is further a problem to obtain an antibody which exhibits good stability and has potential for being used as a drug in terms of efficacy and safety.

Without being bound by theory it appears to be surprising that by changing the LFR regions it is possible to keep the CDR regions closer to the non-humanized murine antibody, thus retaining the affinity, and still allowing a high degree of humanization of the antibody.

According to an embodiment, the invention concerns a humanized antibody or antigen binding fragment thereof, capable of binding to B7H3 antigen, comprising

regions having a total at least 90% identity to SEQ ID No. 28-30, 67-69, 73-75, 79-81, 85-87, 91-93,

regions having a total at least 90% identity to SEQ ID No. 25-27, 64-66, 70-72, 76-78, 82-84, 88-90, 94-99,

regions having a total at least 75% identity to the sequences according to SEQ ID No. 40-43, 48-51, 56-59 or 108-119, and

regions having a total at least 75% identity to the sequences according to SEQ ID No. 44-47, 52-55, 60-63 or 120-131.

According to an embodiment, the invention concerns a humanized antibody or antigen binding fragment thereof, comprising

regions having a total at least 90%, more preferred 95% identity to SEQ ID No. 28-30, 67-69, 73-75, 79-81, 85-87, 91-93, and/or

regions having a total at least 75% identity to SEQ ID No. 40-43, 48-51, 56-59 or 108-119.

According to an embodiment, the invention concerns a humanized antibody or antigen binding fragment thereof, comprising

regions having a total at least 80%, more preferred 85, preferably 90%, more preferred 95% identity to SEQ ID No. 40-43, 48-51, 56-59 or 108-119.

According to an embodiment, the invention concerns a humanized antibody or antigen binding fragment thereof, comprising

regions having a total at least 90%, more preferred 95% identity to SEQ ID No. 25-27, 64-66, 70-72, 76-78, 82-84, 88-90, 94-99, and/or

regions having a total at least 75% identity to SEQ ID No. 44-47, 52-55, 60-63 or 120-131.

According to an embodiment, the invention concerns a humanized antibody or antigen binding fragment thereof, comprising

regions having a total at least 80%, more preferred 85, preferably 90%, more preferred 95% identity to SEQ ID No. 44-47, 52-55, 60-63 or 120-131.

According to an embodiment, the invention concerns a humanized antibody or antigen binding fragment thereof, comprising

regions selected among the SEQ ID No. 25-30 and 64-99.

According to an embodiment, the invention concerns a humanized antibody or antigen binding fragment thereof, comprising

regions selected among SEQ ID No. 28-30, 67-69, 73-75, 79-81, 85-87, 91-93,

regions selected among SEQ ID No. 25-27, 64-66, 70-72, 76-78, 82-84, 88-90, 94-99,

regions selected among SEQ ID No. 40-43, 48-51, 56-59 or 108-119, and

regions selected among SEQ ID No. 44-47, 52-55, 60-63, or 120-131.

According to an embodiment, the invention concerns a humanized antibody or antigen binding fragment thereof, comprising Fc, Fc2 or Null-Fc.

According to an embodiment, the invention concerns a humanized antibody or antigen binding fragment thereof, capable of binding to B7H3 antigen, wherein said antibody or antigen binding fragment comprises sequences having at least 70% identity to sequences selected among any of the sequences SEQ ID No. 36, 37, 38 and 39.

According to an embodiment, the invention concerns the antibody or antigen binding fragment thereof according to the invention, wherein said antibody or antigen binding fragment comprises sequences having at least 75% identity to sequences selected among any of the sequences SEQ ID No. 36, 37, 38 or 39.

According to an embodiment the invention concerns the antibody or antigen binding fragment according to the invention, wherein said antibody or antigen binding fragment comprises at least one sequence selected among a heavy chain variable region CDR1 according to SEQ ID No. 25, 64, 70, 76, 82, 88, 94 and 97 a heavy chain variable region CDR2 according to SEQ IN No. 26, 65, 71, 77, 83, 89, 95 and 98 a heavy chain variable region CDR3 according to SEQ IN No. 27, 66, 72, 78, 84, 90, 96 and 99 a light chain variable region CDR1 according to SEQ ID No. 28, 67, 73, 79, 85 and 91 a light chain variable region CDR2 according to SEQ ID No. 29, 68, 74, 80, 86 and 92 and a light chain variable region CDR3 according to SEQ ID No. 30, 69, 75, 81, 87 and 93.

According to an embodiment, the invention concerns the antibody or antigen binding fragment according to the invention, wherein said antibody comprises a heavy chain sequence according to SEQ ID No. 1, 2, 3, 4, 5, 6, 7, 8 or 9 and/or a light chain sequence according to SEQ ID No. 10, 11, 12, 13 or 14.

According to an embodiment, the invention concerns the antibody or antigen binding fragment, wherein said antibody comprises a heavy chain sequence that is at least about 80%, about 81%, about 82%, about 83%, about 84%, about 85%, about 86%, about 87%, about 88%, about 89%, about 90%, about 91% about 92%, about 93%, about 94%, about 95%, about 96%, about 97%, about 98% or about 99% sequence identity to the sequence set forth in SEQ ID No. 1, 2, 3, 4, 5, 6, 7, 8 or 9 and/or a light chain sequence that is at least about 80%, about 81%, about 82%, about 83%, about 84%, about 85%, about 86%, about 87%, about 88%, about 89%, about 90%, about 91% about 92%, about 93%, about 94%, about 95%, about 96%, about 97%, about 98% or about 99% sequence identity to the sequence set forth in SEQ ID No. 10, 11, 12, 13 or 14.

According to an embodiment, the invention concerns the antibody or antigen binding fragment, wherein said antibody or antigen binding fragment comprises at least 50% amino acids that are identical to the amino acids of the human germline of said antibody or antigen binding fragment, at least 55%, at least 60%, at least 65%, at least 70%, at least 75%, at least 77.5%, at least 80%, at least 82%, at least 84%, at least 86%, at least 88% or at least 90%.

According to an embodiment, the invention concerns the antibody or antigen binding fragment, wherein said antibody or antigen binding fragment binds to an epitope, and wherein said epitope is an epitope of B7H3.

According to an embodiment, the invention concerns the antibody or antigen binding fragment, wherein antibody of antigen binding fragment binds to the sequence according to SEQ ID No.: 33.

According to an embodiment, the invention concerns the antibody or antigen binding fragment, wherein said antibody or antigen binding fragment binds to an antigen, and wherein said antigen comprises a sequence selected among SEQ ID No. 31 and 32.

According to an embodiment, the invention concerns the antibody or antigen binding fragment, wherein said antigen is present on a cancer cell.

According to an embodiment, the invention concerns the antibody or antigen binding fragment according to the invention, wherein said cancer cells is from a metastasis.

According to an embodiment, the invention concerns the antibody or antigen binding fragment according to the invention, wherein said cancer cells and/or metastasis is prostate cancer, a desmoplastic small round cell tumor, ovarian cancer, gastric cancer, pancreatic cancer, liver cancer, renal cancer, breast cancer, non-small cell lung cancer, melanoma, alveolar rhabdomyosarcoma, embryonal rhabdomyosarcoma, Ewing sarcoma, Wilms tumor, neuroblastoma, ganglioneuroblastoma, ganglioneuroma, medulloblastoma, high-grade glioma, diffuse intrinsic pontine glioma, embryonal tumors with multilayered rosettes, or a cancer expressing B7H3.

According to an embodiment, the invention concerns the antibody or antigen binding fragment, comprising a Fc region which does not interact with a Fc gamma receptor.

According to an embodiment, the invention concerns the antibody or antigen binding fragment, further comprising an Fc region, wherein said Fc region is not reactive or exhibit little reactivity.

According to an embodiment, the invention concerns the antibody or antigen binding fragment, wherein said antibody comprises a null Fc.

According to an embodiment, the invention concerns the antibody or antigen binding fragment thereof, wherein said antibody or antigen binding fragment has an immunogenicity of less than 50%, less than 45%, less than 40%, less than 35%, less than 30%, less than 25%, less than 20%, less than 15% or about 10%.

According to an embodiment, the invention concerns the antibody or antigen binding fragment thereof, wherein said agent or antibody is a murine antibody or an antigen binding fragment thereof.

According to an embodiment, the invention concerns the antibody or antigen binding fragment thereof, wherein said agent or antibody is a chimeric antibody or an antigen binding fragment thereof.

According to an embodiment, the invention concerns the antibody or antigen binding fragment thereof, wherein said agent or antibody is a humanized antibody or an antigen binding fragment thereof.

According to an embodiment, the invention concerns the antibody or antigen binding fragment thereof, wherein said antibody or antigen binding fragment is radiolabeled with a radioactive isotope.

According to an embodiment, the invention concerns the antibody or antigen binding fragment thereof, wherein said radioactive isotope is selected among a PET label and or a SPECT label.

According to an embodiment, the invention concerns the antibody or antigen binding fragment thereof, wherein said PET label is selected among ¹²⁴I, ²²⁵Ac and ⁸⁹Zr.

According to an embodiment, the invention concerns the antibody or antigen binding fragment thereof, wherein said SPECT label is selected among ¹³¹I, ¹⁷⁷Lu, ⁹⁹mTc, ⁶⁴Cu and ⁸⁹Zr.

According to an embodiment, the invention concerns the antibody or antigen binding fragment thereof, wherein said antibody or antigen binding fragment is conjugated to a chelator compound.

According to an embodiment, the invention concerns the antibody or antigen binding fragment thereof, wherein said chelator compound is bound to a radioactive isotope.

According to an embodiment, the invention concerns the antibody or antigen binding fragment thereof, wherein said radioactive isotope is selected among ¹²⁴I, ¹³¹I and ¹⁷⁷Lu or ⁹⁹mTc, ⁶⁴Cu and ⁸⁹Zr.

According to an embodiment, the invention concerns the antibody or antigen binding fragment thereof, wherein said chelator compound is selected among DOTA, DTPA, NOTA and DFO.

According to an embodiment, the invention concerns the antibody or antigen binding fragment thereof, wherein said DOTA is a variant of DOTA, such as Benzyl-DOTA.

According to an embodiment, the invention concerns the antibody or antigen binding fragment thereof, wherein said DTPA is a variant of DTPA, such as CHX-A″-DTPA.

According to an embodiment, the invention concerns the antibody or antigen binding fragment, wherein said radioactive isotope is an alpha, beta or positron emitting radionuclide.

According to an embodiment, the invention concerns the antibody or antigen binding fragment, comprising a structure selected among IgG, IgG1, IgG2, IgG3, and IgG4.

According to an embodiment, the invention concerns the antibody or antigen binding fragment, comprising a structure selected among IgG, IgM, IgA, IgD, and IgE.

According to an embodiment, the invention concerns an antibody or antigen binding fragment thereof, capable of binding to an antigen, wherein said antibody or antigen binding fragment comprises a sequence according to SEQ ID No. 15, 16 17 or 132.

According to an embodiment, the invention concerns a self-assembly disassembly (SADA) polypeptide, wherein said polypeptide is linked to an antibody or antigen binding fragment according to the invention.

According to an embodiment, the invention concerns the antibody or antigen binding fragment, wherein said antibody or antigen binding fragment thereof is a bispecific and/or trispecific binding antibody.

According to an embodiment, the invention concerns the antibody or antigen binding fragment, wherein said bispecific and/or trispecific binding antibody comprises a first antibody or antigen binding fragment thereof according to the invention for binding to a first antigen, and a second antibody or antigen binding fragment for binding to a second antigen.

According to an embodiment, the invention concerns the antibody or antigen binding fragment, wherein said second antibody or antigen binding fragment thereof binds to DOTA and/or DTPA.

DOTA (Dodecane Tetraacetic Acid) is also referred to as 1,4,7,10-tetraazacyclododecane-1,4,7 10-tetraacetic acid, and has the formula (CH₂CH₂NCH₂CO₂H)₄.

DTPA (Diethylene Triamine Pentaacetic Acid) is also referred to with the IUPAC name 2-[bis[2-[bis(carboxymethyl)amino]ethyl]amino]acetic acid. DTPA has the molecular formula C14H23N3O10.

According to an embodiment, the invention concerns the antibody or antigen binding fragment, wherein said antibody or antigen binding fragment is linked to a self-assembly disassembly (SADA) polypeptide.

In certain embodiments, the antibody of antigen binding fragment thereof is linked to a self-assembly disassembly (SADA) polypeptide disclosed in International Patent Application Publication No. WO2018204873, all of which is incorporated by reference in its entirety.

According to an embodiment, the invention concerns the antibody or antigen binding fragment, wherein said self-assembly disassembly (SADA) polypeptide has an amino acid sequence that shows at least 75% identity with that of a human homo-multimerizing polypeptide and being characterized by one or more multimerization dissociation constants (KD).

According to an embodiment, the invention concerns a polypeptide conjugate comprising:

a self-assembly disassembly (SADA) polypeptide according to the invention and an antibody or antigen binding fragment according to the invention.

According to an embodiment, the invention concerns a polypeptide conjugate comprising a self-assembly disassembly (SADA) polypeptide, and wherein said conjugate further comprises the bispecific antibody according to the invention, wherein said first antigen is B7H3 and wherein said second antigen is DOTA.

According to an embodiment, the invention concerns a polypeptide conjugate comprising a self-assembly disassembly (SADA) polypeptide, and at least a first binding domain that binds to a first target and is covalently linked to the SADA polypeptide.

According to an embodiment, the invention concerns the polypeptide conjugate, wherein said self-assembly disassembly (SADA) polypeptide has an amino acid sequence that shows at least 75% identity with that of a human homo-multimerizing polypeptide and being characterized by one or more multimerization dissociation constants (KD); and wherein said conjugate is being constructed and arranged so that it adopts a first multimerization state and one or more higher-order multimerization states, wherein: the first multimerization state is less than about −70 kDa in size, at least one of the higher-order multimerization states is a homo-tetramer or higher-order homo multimer greater than 150 kDa in size, wherein the higher-order homo-multimerized conjugate is stable in aqueous solution when the conjugate is present at a concentration above the SADA polypeptide KD, and the conjugate transitions from the higher-order multimerization state(s) to the first multimerization state under physiological conditions when the concentration of the conjugate is below the SADA polypeptide KD.

According to an embodiment, the invention concerns the polypeptide conjugate, wherein said conjugate comprises a chelator.

According to an embodiment, the invention concerns the conjugate wherein said chelator comprises a metal ion.

According to an embodiment, the invention concerns the conjugate, wherein the metal ion is a radionuclide.

According to an embodiment, the invention concerns an isolated nucleic acid molecule encoding the antibody or antigen binding fragment of the invention.

According to an embodiment, the invention concerns an isolated nucleic acid molecule comprising a sequence according to SEQ ID No. 18, 19, 20, 21 or 22.

According to an embodiment, the invention concerns a recombinant vector comprising the isolated nucleic acid molecule of the invention.

According to an embodiment, the invention concerns a host cell comprising the recombinant vector according to the invention.

According to an embodiment, the invention concerns a method for the production of an antibody or antigen binding fragment thereof according to the invention comprising a step of culturing the host cell according to the invention in a culture medium under conditions allowing the expression of the antibody or fragment and separating the antibody or fragment from the culture medium.

According to an embodiment, the invention concerns a chimeric antigen receptor (CAR) comprising an antibody or antigen binding fragment according to the invention.

According to an embodiment, the invention concerns a CAR-T cell expressing a CAR.

According to an embodiment, the invention concerns a population of CAR-T cells.

According to an embodiment, the invention concerns a composition comprising the population of CAR-T cells.

According to an embodiment, the invention concerns a CAR-NK cell expressing a CAR.

According to an embodiment, the invention concerns a population of CAR-NK cells.

According to an embodiment, the invention concerns a composition comprising the population of CAR-NK cells.

According to an embodiment, the invention concerns a pharmaceutical composition comprising the antibody or antigen binding fragment according to the invention.

According to an embodiment, the invention concerns a T cell armed with the antibody or antigen binding fragment according to the invention.

According to an embodiment, the invention concerns a method of treating, preventing, alleviating and/or diagnosing the symptoms of a medical condition in a subject, comprising a step of administration of an antibody, an antigen binding fragment, a bispecific antibody, a trispecific antibody, a polypeptide conjugate, a composition and/or a CAR into the peritoneum, and wherein said medical condition is characterized by expression of B7H3 antigen.

According to an embodiment, the invention concerns a method of imaging a tumor in the peritoneum, wherein said imaging comprises use of an antibody or antigen binding fragment thereof, wherein said tumor is characterized by expression of B7H3 antigen, and wherein the method of imaging has been preceded by a step of administration of an antibody or antigen binding fragment thereof into the peritoneum.

According to an embodiment, the invention concerns the method, wherein said antibody, antigen binding fragment, bispecific antibody, trispecific antibody, polypeptide conjugate, composition and/or CAR is the antibody, antigen binding fragment, bispecific antibody, trispecific antibody, polypeptide conjugate, composition and/or CAR according to the invention.

According to an embodiment, the invention concerns the method, wherein said antibody or antigen binding fragment thereof comprises at least one sequence selected among a heavy chain variable region CDR1 according to SEQ ID No. 25, a heavy chain variable region CDR2 according to SEQ IN No. 26, a heavy chain variable region CDR3 according to SEQ IN No. 27, a light chain variable region CDR1 according to SEQ ID No. 28, a light chain variable region CDR2 according to SEQ ID No. 29 and a light chain variable region CDR3 according to SEQ ID No. 30.

According to an embodiment, the invention concerns the method, wherein said antibody comprises a heavy chain sequence according to SEQ ID No. 1, 2, 3, 4, 5, 6, 7, 8, 9 or 23 and/or a light chain sequence according to SEQ ID No. 10, 11, 12, 13, 14 or 24.

According to an embodiment, the invention concerns the method, wherein antibody comprises a heavy chain sequence that is at least about 80%, about 81%, about 82%, about 83%, about 84%, about 85%, about 86%, about 87%, about 88%, about 89%, about 90%, about 91% about 92%, about 93%, about 94%, about 95%, about 96%, about 97%, about 98% or about 99% sequence identity to the sequence set forth in SEQ ID No. 1, 2, 3, 4, 5, 6, 7, 8, 9 or 23 and/or a light chain sequence that is at least about 80%, about 81%, about 82%, about 83%, about 84%, about 85%, about 86%, about 87%, about 88%, about 89%, about 90%, about 91% about 92%, about 93%, about 94%, about 95%, about 96%, about 97%, about 98% or about 99% sequence identity to the sequence set forth in SEQ ID No. 10, 11, 12, 13, 14 or 24.

According to an embodiment, the invention concerns use of the composition according to the invention in the manufacturing of a medicament for the treatment of a cancer, for use in a method according to the invention.

According to an embodiment, the invention concerns use of the antibody of antigen binding fragment according to the invention in the manufacturing of a medicament for the treatment of a cancer and/or for use in a method according to the invention.

According to an embodiment, the invention concerns an in vitro use of an antibody or antigen binding fragment thereof according to the invention.

According to an embodiment, the invention concerns the method, wherein said medical condition is a cancer.

According to an embodiment, the invention concerns the method, wherein said cancer and/or said tumor is a metastasis.

According to an embodiment, the invention concerns the method, wherein said cancer, said tumor and/or said metastasis is prostate cancer, a desmoplastic small round cell tumor, ovarian cancer, gastric cancer, pancreatic cancer, liver cancer, renal cancer, breast cancer, non-small cell lung cancer, melanoma, alveolar rhabdomyosarcoma, embryonal rhabdomyosarcoma, Ewing sarcoma, Wilms tumor, neuroblastoma, ganglioneuroblastoma, ganglioneuroma, medulloblastoma, high-grade glioma, diffuse intrinsic pontine glioma, embryonal tumors with multilayered rosettes, or a cancer expressing B7H3.

According to an embodiment, the invention concerns a method of treating, preventing, alleviating and/or diagnosing the symptoms of a medical condition in a subject, comprising a step of administration of an antibody or antigen binding fragment according to the invention, into the peritoneum, and wherein said medical condition is characterized by expression of B7H3 antigen.

According to an embodiment, the invention concerns a method of imaging a tumor in the peritoneum, wherein said imaging comprises use of an antibody or antigen binding fragment thereof according to the invention, wherein said medical tumor is characterized by expression of B7H3 antigen, and wherein the method of imaging has been preceded by a step of administration of an antibody or antigen binding fragment thereof according to the invention into the peritoneum.

In order to provide a clear and consistent understanding of the specification and claims, including the scope to be given such terms, the following definitions are provided.

Affinity: As is known in the art, “affinity” is a measure of the tightness with which a particular ligand (e.g., an antibody) binds to its partner (e.g., an epitope). Affinities can be measured in difference ways.

Antibody: The term “antibody” is art-recognized terminology and is intended to include molecules or active fragments of molecules that bind to known antigens. Examples of active fragments of molecules that bind to known antigens include Fab and F(ab′)2 fragments. These active fragments can be derived from an antibody of the present invention by a number of techniques. For example, purified monoclonal antibodies can be cleaved with an enzyme, such as pepsin, and subjected to HPLC gel filtration. The appropriate fraction containing Fab fragments can then be collected and concentrated by membrane filtration and the like. The term “antibody” also includes bispecific and chimeric antibodies and other available formats.

Antibody fragment: An antibody fragment is a portion of an antibody such as F(ab′)2, F(ab)2, Fab′, Fab, Fv, sFv and the like. Regardless of structure, an antibody fragment binds with the same antigen that is recognized by the intact antibody. For example, an 3F8 monoclonal antibody fragment binds with an epitope recognized by 3F8. The term “antibody fragment” also includes any synthetic or genetically engineered protein that acts like an antibody by binding to a specific antigen to form a complex. For example, antibody fragments include isolated fragments consisting of the variable regions, such as the “Fv” fragments consisting of the variable regions of the heavy and light chains, recombinant single chain polypeptide molecules in which light and heavy variable regions are connected by a peptide linker (“scFv proteins”), and minimal recognition units consisting of the amino acid residues that mimic the hypervariable region.

Bispecific antibody: A bispecific antibody is an antibody that can bind simultaneously to two targets which are of different structure. Bispecific antibodies (bsAb) and bispecific antibody fragments (bsFab) have at least one arm that specifically binds to an antigen, for example, GD2 and at least one other arm that specifically binds to another antigen, for example a targetable conjugate that bears a therapeutic or diagnostic agent. A variety of bispecific fusion proteins can be produced using molecular engineering. In one form, the bispecific fusion protein is divalent, consisting of, for example, a scFv with a single binding site for one antigen and a Fab fragment with a single binding site for a second antigen. In another form, the bispecific fusion protein is tetravalent, consisting of, for example, an IgG with two binding sites for one antigen and two identical scFv for a second antigen.

Chimeric antibody: A chimeric antibody is a recombinant protein that contains the variable domains including the complementarity-determining regions (CDRs) of an antibody derived from one species, for example a rodent antibody, while the constant domains of the antibody molecule is derived from those of a human antibody. The constant domains of the chimeric antibody may also be derived from that of other species, such as a cat or dog.

Effective amount: As used herein, the term “effective amount” refers to an amount of a given compound, conjugate or composition that is necessary or sufficient to realize a desired biologic effect. An effective amount of a given compound, conjugate or composition in accordance with the methods of the present invention would be the amount that achieves this selected result, and such an amount can be determined as a matter of routine by a person skilled in the art, without the need for undue experimentation.

Humanized antibody: A humanized antibody is a recombinant protein in which the CDRs from an antibody from one species; e.g., a rodent antibody, is transferred from the heavy and light variable chains of the rodent antibody into human heavy and light variable domains. The constant domain of the antibody molecule is derived from those of a human antibody.

A human antibody may be an antibody obtained from transgenic mice that have been “engineered” to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas.

Immunogenicity: Immunogenicity may be defined as the propensity of a therapeutic protein product to generate immune responses to itself and to related proteins or to induce immunologically related adverse clinical events (FDA). Immunogenicity may refer to the ability of a molecule or substance to provoke an immune response. Unwanted immunogenicity may be an immune response by an organism against a therapeutic antigen. The frequency of anti-therapeutic antibody response in patients is described as % of the patient group.

Immunogenicity may be tested by ELISA for HAMA (Human AntiMouse Antibodies). In this technique serum samples are analyzed via an established ELISA assay using mouse antibody as capture antigen.

Prevent: As used herein, the terms “prevent”, “preventing” and “prevention” refer to the prevention of the recurrence or onset of one or more symptoms of a disorder in a subject as result of the administration of a prophylactic or therapeutic agent.

Radioactive isotope: Examples of radioactive isotopes that can be conjugated to antibodies for use diagnostically or therapeutically include, but are not limited to, ²¹¹At, ¹⁴C, ⁵¹Cr, ⁵⁷Co, ⁵⁸Co, ⁶⁷CU, ¹⁵²Eu, ⁶⁷Ga, ³H, ¹¹¹In, ⁵⁹Fe, ²¹²Pb, ¹⁷⁷Lu, ³²P, ²²³Ra, ²²⁴Ra, ¹⁸⁶Re, ¹⁸⁸Re, ⁷⁵Se, ³⁵S, ⁹⁹mTc, ²²⁷Th, ⁸⁹Zr, ⁹⁰Y, ¹²³I, ¹²⁴I, ¹²⁵I, ¹³¹I, ^{94m}Tc, ⁶⁴Cu, ⁶⁸Ga, ⁶⁶Ga, ⁷⁶Br, ⁸⁶Y, ⁸²Rb, ^{110m}In, ¹³N, ¹¹C, ¹⁸F and alpha-emitting particles. Non-limiting examples of alpha-emitting particles include ²⁰⁹Bi, ²¹¹Bi, ²¹²Bi, ²¹³Bi, ²¹⁰Po, ²¹¹Po, ²¹²Po, ²¹⁴Po, ²¹⁵Po, ²¹⁶Po, ²¹⁸Po, ²¹¹At, ²¹⁵At, ²¹⁷At, ²¹⁸At, ²¹⁸Rn, ²¹⁹Rn, ²²⁰Rn, ²²²Rn, ²²⁶Rn, ²²¹Fr, ²²³Ra, ²²⁴Ra, ²²⁶Ra, ²²⁵Ac, ²²⁷Ac, ²²⁷Th, ²²⁸Th, ²²⁹Th, ²³⁰Th, ²³²Th, ²³¹Pa, ²³³U, ²³⁴U, ²³⁵U, ²³⁶U, ²³⁸U, ²³⁷Np, ²³⁸Pu, ²³⁹Pu, ²⁴⁰Pu, ²⁴⁴Pu, ²⁴¹Am, ²⁴⁴Cm, ²⁴⁵Cm, ²⁴⁸Cm, ²⁴⁹Cf, and ²⁵²Cf.

Sequence Alignment: Sequence alignment refers simply to any way to align two sequences one below another. It is a way of arranging sequences of DNA, RNA or protein to identify regions of similarity between the sequences. Different alignment algorithms exists and they usually have a scoring function which assigns every alignment a numeric score indicating how good an alignment is and tries to find the best alignment according to its scoring function.

Sequence Identity: The term “Sequence identity” is used herein as a measurement of the relatedness of two amino acid or nucleic acid sequences. In order to calculate the sequence identity between two sequences the sequences are aligned and the longest overlap identified. Sequence identity is calculated as the percentage of identical residues in corresponding positions in the overlap of the total length of the overlap.

A variety of computational algorithms are available for the skilled person, for generating sequence alignment and calculating Sequence identity. As used herein, Sequence alignment refers to Pairwise alignments. Several algorithms perform this including the sequence alignment program FASTA, which uses the Smith-Waterman algorithm.

As used herein the sequence alignment may refer to the following algorithm and parameters:

Algorithm: FASTA (3.8 Nov. 2011) [optimized]

Parameters: BL50 matrix (15:−5), open/ext: −10/−2

ktup: 2, E-join: 1 (1), E-opt: 0.2 (1), width: 16

Subject: By “subject” or “individual” or “animal” or “patient” or “mammal,” is meant any subject, particularly a mammalian subject, for whom diagnosis, prognosis, or therapy is desired. Mammalian subjects include humans and other primates, domestic animals, farm animals, and zoo, sports, or pet animals such as dogs, cats, guinea pigs, rabbits, rats, mice, horses, cattle, cows, and the like.

Treatment: As used herein, the terms “treatment,” “treat,” “treated” or “treating” refer to prophylaxis and/or therapy, particularly wherein the object is to prevent or slow down (lessen) an undesired physiological change or disorder, such as the progression of multiple sclerosis. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

### FIGURES

FIG. 1 shows SEC-HPLC results for Chimeric IgG

FIG. 2 shows SEC-HPLC results for L2H3

FIG. 3 shows SEC-HPLC results for L2H4

FIG. 4 shows SEC-HPLC results for L2H5

FIG. 5 shows schematic design of SADA. The tetramerization domain connected to ScFv against DOTA via a linker, the ScFv against DOTA connected via a second linker to ScFv against B7-H3

FIG. 6 shows binding of SADA to ovarian cancer and glioblastoma cell lines

All cited references are incorporated by reference.

The accompanying Figures and Examples are provided to explain rather than limit the present invention. It will be clear to the person skilled in the art that aspects, embodiments, claims and any items of the present invention may be combined.

Unless otherwise mentioned, all percentages are in weight/weight. Unless otherwise mentioned, all measurements are conducted under standard conditions (ambient temperature and pressure). Unless otherwise mentioned, test conditions are according to European Pharmacopoeia 8.0.

### EXAMPLES

### Example 1: Designing Humanized Anti-B7-H3 Antibodies

Murine 8H9 antibody comprising a heavy chain sequence according to SEQ ID No. 23 and a light chain sequence according to SEQ ID No. 24 was humanized by grafting the IMGT CDR residues onto a human IgG1 backbone, using the human germline sequences IGKV6-21*02 (SEQ ID No. 37) for the kappa variable domain and IGHV1-8*01 (SEQ ID No. 38) for the variable heavy domain. Select back mutations were then inserted based on rational design and molecular modelling of the crystal structure of the murine 8H9 antibody Fab fragment comprising a heavy chain sequence according to SEQ ID No. 34 and a light chain sequence according to SEQ ID No. 35 (Protein Data Bank structure SCMA: Anti-B7H3 monoclonal antibody ch8H9 Fab fragment) to generate 6 humanized VH candidates and 4 humanized VL candidates, each of which had a human germline content of >85%. Computational modelling was done using Biovia Discovery Studio software (Dassault Systemes). Each of the humanized sequences were rationally engineered to retain the affinity of murine 8H9.

### Example 2: Generation and Characterization of Humanized Anti-B7-H3 Antibodies

Chimeric and humanized antibodies were generated using CDR grafting methods using closely homologous human germline sequences. Six different humanized VH and four humanized VL sequences were combined to generate 24 different humanized IgG1 antibodies. The antibodies were expressed in HEK293 cells and purified using Protein A resin. Antibodies were buffer exchanged into PBS and quantified by OD280 and titer was calculated. Sample purity and aggregation status was determined by analytical Size Exclusion Chromatography (SEC-HPLC), which separates molecules based on molecular mass and hydrodynamic volume. Area % of each peak is calculated based on total area of the peaks. Peak symmetry is calculated to determine the peak fronting (<1) or tailing (>1). A column of 300A pore size was used for detection at 280 nm and PBS as the running buffer. Surface plasmon resonance studies to assess binding affinities were carried out on a Carterra LSA instrument. An anti-human Fc antibody “lawn” was prepared on a HC30M chip by amine coupling. The chip was activated with equal volume of 100 mM MES pH 5.5, 100 mM S-NHS and 400 mM EDC. The anti-human IgG Fc antibody was immobilized. The chip was deactivated with 1 M ethanolamine pH 8.5. Antibodies were diluted and printed onto the anti-human Fc lawn. An 8-point series of 4Ig human B7-H3 was prepared. Association was observed for 5 minutes and dissociation was observed for 15 minutes for kinetic study.

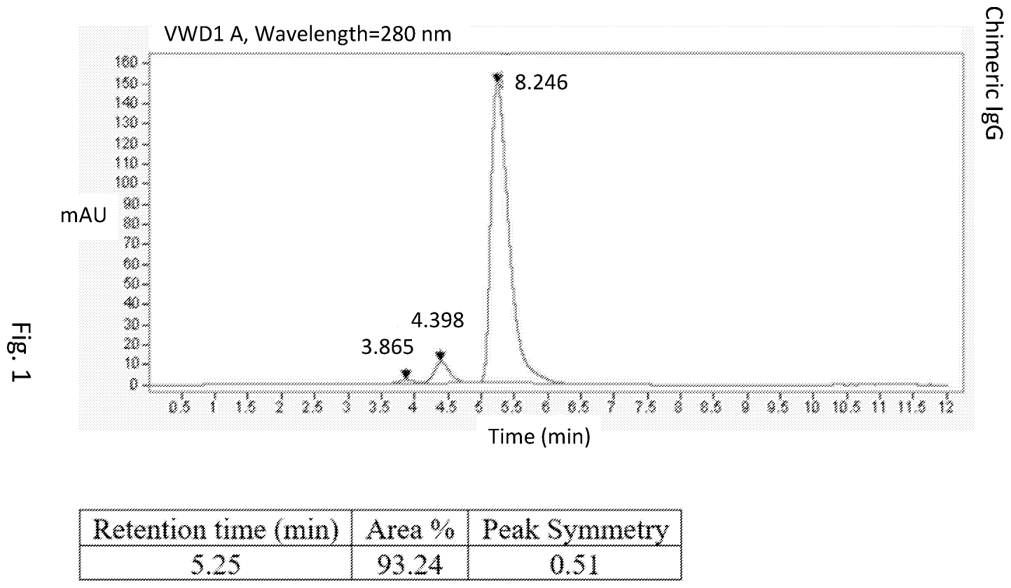

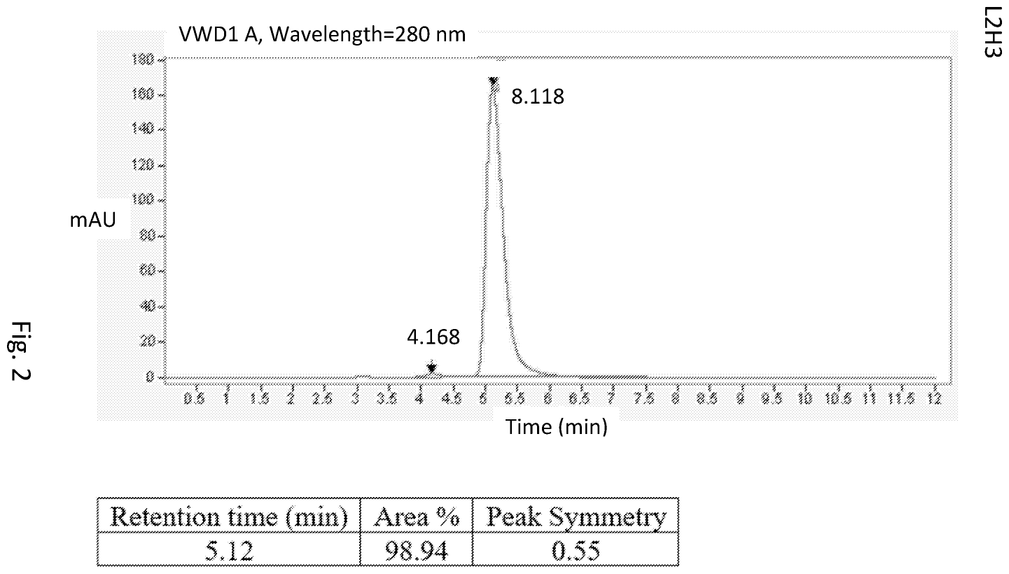

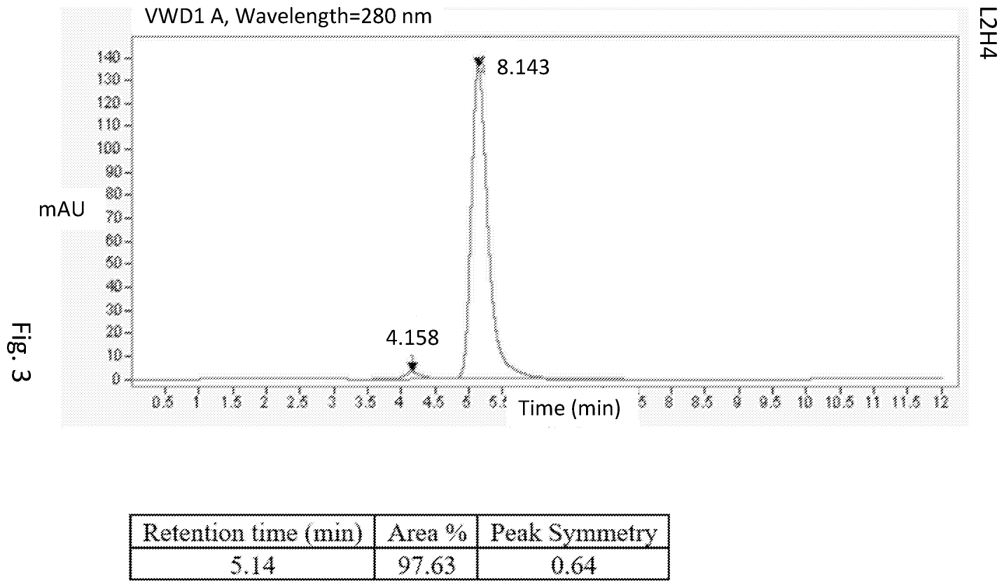

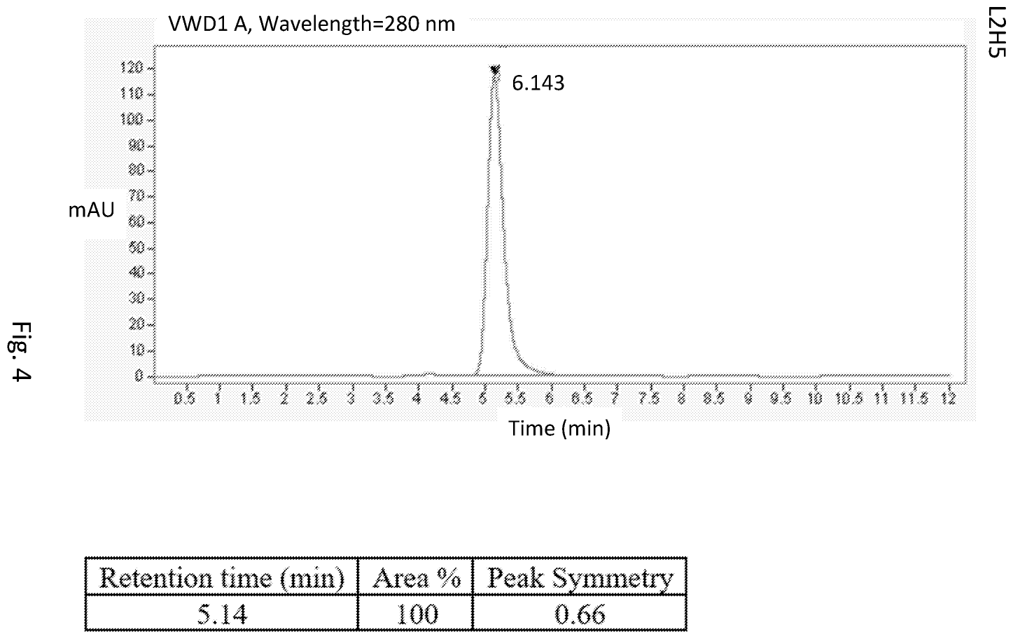

The characterization data, including expression titers (mg of purified protein per L of culture), purity (% monomer peak by analytical SEC-HPLC) and affinity measurement by SPR (ka, kd and K_{D}) are summarized in Table 1 and FIG. 1-4.

TABLE 1
Characterization of chimeric and humanized anti-B7H3 antibodies

| Antibody | Titer | HPLC | ka | kd | K_{D} |
|---|---|---|---|---|---|
| Name | (mg/L) | peak % | (M⁻¹ s⁻¹) | (s⁻¹) | (M) |
| chimeric | 365.16 | 93.24 | 7.8E+04 | 7.9E−04 | 1.0E−08 |
| L1H1 | 617.23 | 100.00 | 3.1E+04 | 4.3E−04 | 1.5E−08 |
| L1H2 | 470.88 | 96.95 | 2.9E+04 | 4.1E−04 | 1.4E−08 |
| L1H3 | 518.95 | 100.00 | 4.3E+04 | 3.5E−04 | 8.1E−09 |
| L1H4 | 596.64 | 96.30 | 4.3E+04 | 4.1E−04 | 9.5E−09 |
| L1H5 | 524.47 | 100.00 | 3.9E+04 | 4.2E−04 | 1.1E−08 |
| L1H6 | 297.43 | 93.60 | 2.6E+04 | 6.6E−04 | 2.5E−08 |
| L2H1 | 618.12 | 100.00 | 3.9E+04 | 4.1E−04 | 1.1E−08 |
| L2H2 | 493.54 | 98.52 | 3.3E+04 | 3.7E−04 | 1.1E−08 |
| L2H3 | 450.01 | 98.94 | 3.4E+04 | 3.4E−04 | 9.9E−09 |
| L2H4 | 623.94 | 97.63 | 4.4E+04 | 3.6E−04 | 8.2E−09 |
| L2H5 | 533.95 | 100.00 | 4.2E+04 | 4.0E−04 | 9.4E−09 |
| L2H6 | 365.41 | 95.75 | 2.1E+04 | 5.1E−04 | 2.4E−08 |
| L3H1 | 479.33 | 98.07 | 3.4E+04 | 3.4E−04 | 1.0E−08 |
| L3H2 | 275.53 | 96.05 | 3.2E+04 | 3.5E−04 | 1.1E−08 |
| L3H3 | 396.32 | 98.45 | 3.2E+04 | 3.2E−04 | 9.7E−09 |
| L3H4 | 415.63 | 96.24 | 4.0E+04 | 3.3E−04 | 8.4E−09 |
| L3H5 | 434.70 | 100.00 | 3.8E+04 | 3.5E−04 | 9.2E−09 |
| L3H6 | 94.34 | 89.95 | 2.5E+04 | 5.0E−04 | 2.0E−08 |
| L4H1 | 662.55 | 100.00 | 2.4E+04 | 5.5E−04 | 2.3E−08 |
| L4H2 | 511.13 | 100.00 | 3.6E+04 | 3.9E−04 | 1.1E−08 |
| L4H3 | 522.19 | 100.00 | 3.1E+04 | 3.9E−04 | 1.3E−08 |
| L4H4 | 607.17 | 98.16 | 3.4E+04 | 3.6E−04 | 1.1E−08 |
| L4H5 | 504.25 | 100.00 | 4.3E+04 | 4.0E−04 | 9.3E−09 |
| L4H6 | 434.71 | 96.28 | 4.3E+04 | 4.2E−04 | 9.6E−09 |

The IMGT/DomainGapAlign (imgt.org) tool was used to calculate the human germline content for the variable regions of heavy chains H1-H6 and light chain L1-L4 (Table 2).

TABLE 2
Human germline content of humanized variable regions.

| candidate | % human germline content |
|---|---|
| VH1 | 87.8 |
| VH2 | 85.7 |
| VH3 | 87.8 |
| VH4 | 87.8 |
| VH5 | 89.8 |
| VH6 | 85.7 |
| VL1 | 89.5 |
| VL2 | 89.5 |
| VL3 | 86.3 |
| VL4 | 85.3 |

Based on the expression titers, % purity, binding affinities compared to the chimeric IgG and human germline content, the three constructs L2H3, L2H4 and L2H5 were chosen as the lead candidates. L2H5 had the greatest human content (both VH5 and VL2) and purity (FIG. 1-4) among the three, all of which were higher than the parental chimeric antibody. L2H5 also had nearly identical affinity to the chimeric IgG (9.4 nM and 10 nM K_{D}, respectively). Therefore, L2H5 was selected for further investigation, including the generation of anti-B7H3×anti-DOTA bispecific SADA (self-assembly and disassembly) antibody construct.

### Example 3: Generation and Characterization of Anti-B7-H3 SADAs for Radioimmunotherapy

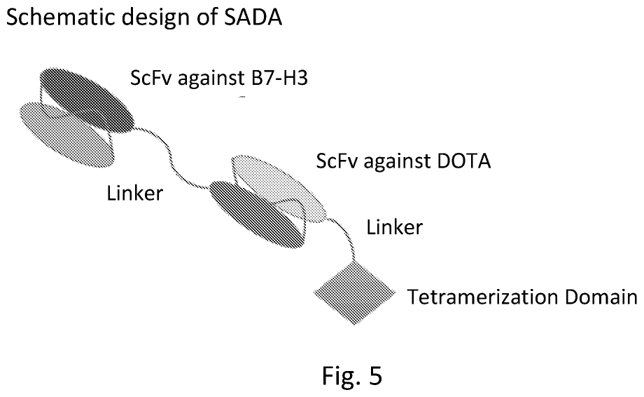

Three anti-B7H3×anti-DOTA bispecific SADA constructs (3BH-1, 3BH-2, 3BH-3) were designed as shown in FIG. 5. 3BH-1 consists of an anti-B7H3 scFv from the mouse 8H9 antibody comprising a heavy chain sequences according to SEQ ID No. 23 and a light chain sequence according to SEQ ID No. 24 in the orientation of VH-VL. 3BH-2 consists of the anti-B7H3 scFv from the L2H5 sequence in the orientation of VH-VL and 3BH-3 has a disulfide bond in the ScFv of L2H5 (Nh44-VL100 by Kabat numbering) for improved stability. All three constructs include the same anti-DOTA scFv (humanized C825) with VH-VL disulfide stabilization.

### Example 4: SADA Binding to B7H3(+) Cell Lines Via Flow Cytometry

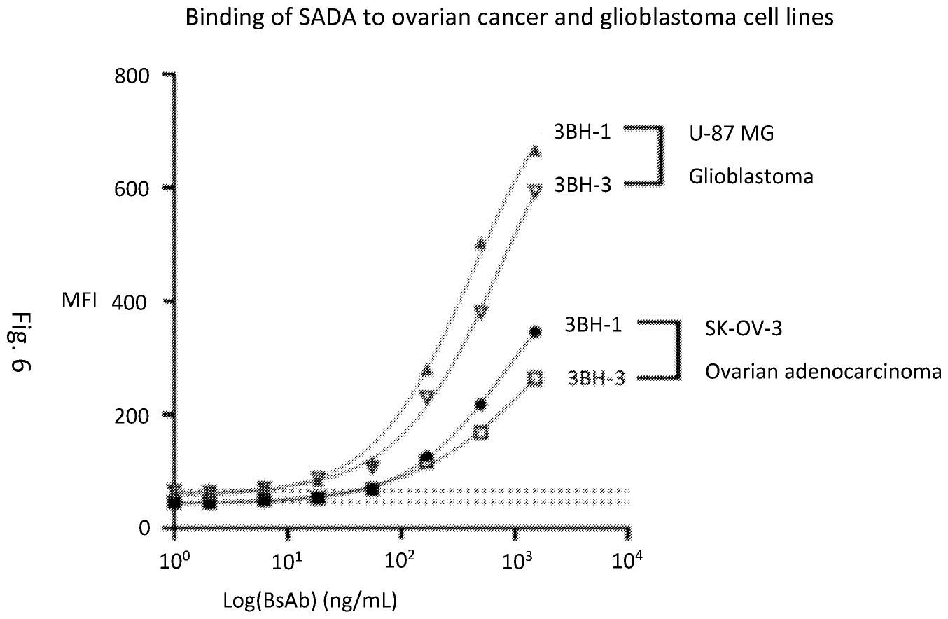

Two cancer cell lines were incubated with SADA proteins at 4° C. for 30 min with a series of titrations separately. After a washing step, the cells were incubated with fluorophore-conjugated secondary antibody (anti-His) at 4° C. for 30 min. After washing again, the cells were analyzed using flow cytometer. 3BH-1 and 3BH-3 have exhibited concentration-dependent binding to glioblastoma cell line U-87 MG and ovarian adenocarcinoma cell line SK-OV-3 (FIG. 6), which are B7-H3 Positive cell lines.

### Example 5: Anti-Tumor Effect of 131I-Labeled Anti-B7-H3 Antibodies In Vivo

To evaluate the anti-tumor effect of the humanized anti-B7H3 antibodies, the luciferase-transfected human ovarian cancer SKOV-3 cells or gastric cancer NCI-N87 can be injected into the abdominal cavities of nude mice. In the planned protocol Iodine-131 (¹³¹I) labeled YB8-L2H5 with null Fc is to be administered as a single injection at different doses and tumor burden is to be assessed weekly using bioluminescence imaging.

Additional relevant sequences are provided below.

SEQ ID NO: 1: Chimeric Heavy Chain (Parental)
QVQLQQSGAELVKPGASVKLSCKASGYTFTNYDINWVRQRPEQGLEWIGWIFPGDGSTQYNEKFKGKA
TLTTDTSSSTAYMQLSRLTSEDSAVYFCARQTTATWFAYWGQGTLVTVSAASTKGPSVFPLAPSSKSTSG
GTAALGCLVKDYFPEPVTVSWNSGALTSGVHTFPAVLQSSGLYSLSSVVTVPSSSLGTQTYICNVNHKPSN
TKVDKKVEPKSCDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGK
SEQ ID NO: 2: YB8 H1
QVQLVQSGAEVKKPGASVKVSCKASGYTFTNYDINWVRQATGQGLEWIGWIFPGDGSTQYNEKFQGR
VTLTTNTSISTAYMELSSLRSEDTAVYYCARQTTATWFAYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPSN
TKVDKKVEPKSCDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGK
SEQ ID NO: 3: YB8 H2
QVQLQQSGAEVKKPGASVKVSCKASGYTFTNYDINWVRQATGQGLEWIGWIFPGDGSTQYNEKFKGR
ATLTRNTSISTAYMELSSLRSEDTAVYYCARQTTATWFAYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPSN
TKVDKKVEPKSCDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGK
SEQ ID NO: 4: YB8 H3
QVQLVQSGAEVKKPGASVKVSCKASGYTFTNYDINWVRQATGQGLEWMGWIFPGDGSTQYNEKFKGR
VTLTRNTSISTAYMELSSLRSEDTAVYFCARQTTATWFAYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPSN
TKVDKKVEPKSCDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGK
SEQ ID NO: 5: YB8 H4
QVQLVQSGAEVKKPGASVKVSCKASGYTFTNYDINWVRQATGQGLEWIGWIFPGDGSTQYNEKFQGR
VTMTTNTSISTAYMELSSLRSEDTAVYFCARQTTATWFAYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPSN
TKVDKKVEPKSCDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGK
SEQ ID NO: 6: YB8 H5
QVQLVQSGAEVKKPGASVKVSCKASGYTFTNYDINWVRQATGQGLEWMGWIFPGDGSTQYNEKFQG
RVTMTTNTSISTAYMELSSLRSEDTAVYYCARQTTATWFAYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGK
SEQ ID NO: 7: YB8 H5 N297A and K322A
QVQLVQSGAEVKKPGASVKVSCKASGYTFTNYDINWVRQATGQGLEWMGWIFPGDGSTQYNEKFQG
RVTMTTNTSISTAYMELSSLRSEDTAVYYCARQTTATWFAYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVVVDVSHEDPEVKFNW
YVDGVEVHNAKTKPREEQYASTYRVVSVLTVLHQDWLNGKEYKCAVSNKALPAPIEKTISKAKGQPREPQ
VYTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGK
SEQ ID NO: 8: YB8_H5 L234A, L235A and K322A
QVQLVQSGAEVKKPGASVKVSCKASGYTFTNYDINWVRQATGQGLEWMGWIFPGDGSTQYNEKFQG
RVTMTTNTSISTAYMELSSLRSEDTAVYYCARQTTATWFAYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPEAAGGPSVFLFPPKPKDTLMISRTPEVTCVVVDVSHEDPEVKFNW
YVDGVEVHNAKTKPREEQYNSTYRVVSVLTVLHQDWLNGKEYKCAVSNKALPAPIEKTISKAKGQPREPQ
VYTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGK
SEQ ID NO: 9: YB8 H6
QVQLQQSGAEVKKPGASVKLSCKASGYTFTNYDINWVRQATGQGLEWIGWIFPGDGSTQYAQKFQGR
ATLTTNTSISTAYMELSSLRSEDTAVYFCARQTTATWFAYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPSN
TKVDKKVEPKSCDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGK
SEQ ID NO: 10: Chimeric Light chain (parental)
DIVMTQSPATLSVTPGDRVSLSCRASQSISDYLHWYQQKSHESPRLLIKYASQSISGIPSRFSGSGSGSDFTL
SINSVEPEDVGVYYCQNGHSFPLTFGAGTKLELKRTVAAPSVFIFPPSDEQLKSGTASVVCLLNNFYPREAK
VQWKVDNALQSGNSQESVTEQDSKDSTYSLSSTLTLSKADYEKHKVYACEVTHQGLSSPVTKSFNRGEC
SEQ ID NO: 11: YB8 L1
EIVLTQSPDFQSVTPKEKVTLTCRASQSISDYLHWYQQKPDQSPKLLIKYASQSISGVPSRFSGSGSGSDFTL
VQWKVDNALQSGNSQESVTEQDSKDSTYSLSSTLTLSKADYEKHKVYACEVTHQGLSSPVTKSFNRGEC
SEQ ID NO: 12: YB8 L2
EIVMTQSPDFQSVTPKEKVTITCRASQSISDYLHWYQQKPDQSPKLLIKYASQSISGVPSRFSGSGSGSDFT
LTINSLEAEDAATYYCQNGHSFPLTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACEVTHQGLSSPVTKSFNRGEC
SEQ ID NO: 13: YB8 L3
EIVMTQSPDFQSVTPKEKVTLTCRASQSISDYLHWYQQKPDQSPKLLIKYASQSISGVPSRFSGSGSGSDFT
KVQWKVDNALQSGNSQESVTEQDSKDSTYSLSSTLTLSKADYEKHKVYACEVTHQGLSSPVTKSFNRGEC
SEQ ID NO: 14: YB8 L4
EIVMTQSPDFQSVTPKEKVTLTCRASQSISDYLHWYQQKPDQSPKLLIKYASQSISGIPSRFSGSGSGTDFT
KVQWKVDNALQSGNSQESVTEQDSKDSTYSLSSTLTLSKADYEKHKVYACEVTHQGLSSPVTKSFNRGEC
SEQ ID NO: 15: 3BH-1
QVQLQQSGAELVKPGASVKLSCKASGYTFTNYDINWVRQRPEQGLEWIGWIFPGDGSTQYNEKFKGKA
TLTTDTSSSTAYMQLSRLTSEDSAVYFCARQTTATWFAYWGQGTLVTVSAGGGGSGGGGSGGGGSGG
GGSGGGGSGGGGSDIVMTQSPATLSVTPGDRVSLSCRASQSISDYLHWYQQKSHESPRLLIKYASQSISGI
PSRFSGSGSGSDFTLSINSVEPEDVGVYYCQNGHSFPLTFGAGTKLELKGGGGSGGGGSGGGGSGGGGS
HVQLVESGGGLVQPGGSLRLSCAASGFSLTDYGVHWVRQAPGKGLEWLGVIWSGGGTAYNTALISRFTI
SRDNSKNTLYLQMNSLRAEDTAVYYCARRGSYPYNYFDAWGCGTLVTVSSGGGGSGGGGSGGGGSGG
GGSGGGGSGGGGSQAVVTQEPSLTVSPGGTVTLTCGSSTGAVTASNYANWVQQKPGQCPRGLIGGHN
NRPPGVPARFSGSLLGGKAALTLLGAQPEDEAEYYCALWYSDHWVIGGGTKLTVLGTPLGDTTHTSGKPL
DGEYFTLQIRGRERFEMFRELNEALELKDAQAGKEPGGSGGAPHHHHHH
SEQ ID NO: 16: 3BH-2
QVQLVQSGAEVKKPGASVKVSCKASGYTFTNYDINWVRQATGQGLEWMGWIFPGDGSTQYNEKFQG
RVTMTTNTSISTAYMELSSLRSEDTAVYYCARQTTATWFAYWGQGTLVTVSSGGGGSGGGGSGGGGSG
GGGSGGGGSGGGGSEIVMTQSPDFQSVTPKEKVTITCRASQSISDYLHWYQQKPDQSPKLLIKYASQSIS
GVPSRFSGSGSGSDFTLTINSLEAEDAATYYCQNGHSFPLTFGQGTKLEIKGGGGSGGGGSGGGGSGGG
GSHVQLVESGGGLVQPGGSLRLSCAASGFSLTDYGVHWVRQAPGKGLEWLGVIWSGGGTAYNTALISR
FTISRDNSKNTLYLQMNSLRAEDTAVYYCARRGSYPYNYFDAWGCGTLVTVSSGGGGSGGGGSGGGGS
GGGGSGGGGSGGGGSQAVVTQEPSLTVSPGGTVTLTCGSSTGAVTASNYANWVQQKPGQCPRGLIGG
HNNRPPGVPARFSGSLLGGKAALTLLGAQPEDEAEYYCALWYSDHWVIGGGTKLTVLGTPLGDTTHTSG
KPLDGEYFTLQIRGRERFEMFRELNEALELKDAQAGKEPGGSGGAPHHHHHH
SEQ ID NO: 17: 3BH-3
QVQLVQSGAEVKKPGASVKVSCKASGYTFTNYDINWVRQATGQCLEWMGWIFPGDGSTQYNEKFQG
RVTMTTNTSISTAYMELSSLRSEDTAVYYCARQTTATWFAYWGQGTLVTVSSGGGGSGGGGSGGGGSG
GGGSGGGGSGGGGSEIVMTQSPDFQSVTPKEKVTITCRASQSISDYLHWYQQKPDQSPKLLIKYASQSIS
GVPSRFSGSGSGSDFTLTINSLEAEDAATYYCQNGHSFPLTFGCGTKLEIKGGGGSGGGGSGGGGSGGG
GSHVQLVESGGGLVQPGGSLRLSCAASGFSLTDYGVHWVRQAPGKGLEWLGVIWSGGGTAYNTALISR
FTISRDNSKNTLYLQMNSLRAEDTAVYYCARRGSYPYNYFDAWGCGTLVTVSSGGGGSGGGGSGGGGS
GGGGSGGGGSGGGGSQAVVTQEPSLTVSPGGTVTLTCGSSTGAVTASNYANWVQQKPGQCPRGLIGG
HNNRPPGVPARFSGSLLGGKAALTLLGAQPEDEAEYYCALWYSDHWVIGGGTKLTVLGTPLGDTTHTSG
KPLDGEYFTLQIRGRERFEMFRELNEALELKDAQAGKEPGGSGGAPHHHHHH
SEQ ID NO: 18: YB8 L2H5 Heavy chain
CAGGTTCAGTTGCAGCAGTCTGGCGCCGAAGTGAAGAAACCTGGCGCCTCCGTGAAGCTGTCCTGC
AAGGCTTCTGGCTACACCTTCACCAACTACGACATCAACTGGGTCCGACAGGCTACCGGACAGGGAC
TCGAATGGATCGGCTGGATCTTTCCTGGCGACGGCTCTACCCAGTACGCCCAGAAATTTCAGGGGAG
AGCTACCCTGACCACCAACACCTCTATCTCCACCGCCTACATGGAACTGTCCAGCCTGAGATCCGAGG
ATACCGCCGTGTACTTCTGTGCCAGACAGACCACCGCCACTTGGTTTGCTTATTGGGGCCAGGGCAC
CCTGGTCACCGTTTCTTCCGCTTCTACCAAGGGACCCAGCGTGTTCCCTCTGGCTCCTTCCAGCAAGTC
TACCTCTGGCGGAACAGCTGCTCTGGGCTGCCTGGTCAAGGACTACTTTCCTGAGCCTGTGACCGTG
TCCTGGAACTCTGGCGCTCTGACATCTGGCGTGCACACCTTTCCAGCTGTGCTGCAGTCCTCCGGCCT
GTACTCTCTGTCCTCTGTCGTGACCGTGCCTTCCAGCTCTCTGGGAACCCAGACCTACATCTGCAATGT
GAACCACAAGCCTTCCAACACCAAGGTGGACAAGAAGGTGGAACCCAAGTCCTGCGACAAGACCCA
CACCTGTCCTCCATGTCCTGCTCCAGAACTGCTCGGCGGACCTTCCGTGTTCCTGTTTCCTCCAAAGCC
TAAGGACACCCTGATGATCTCTCGGACCCCTGAAGTGACCTGCGTGGTGGTGGATGTGTCTCACGAG
GACCCAGAAGTGAAGTTCAATTGGTACGTGGACGGCGTGGAAGTGCACAACGCCAAGACCAAGCCT
AGAGAGGAACAGTACAACTCCACCTACAGAGTGGTGTCCGTGCTGACCGTGCTGCACCAGGATTGG
CTGAACGGCAAAGAGTACAAGTGCAAGGTGTCCAACAAGGCCCTGCCTGCTCCTATCGAAAAGACC
ATCTCCAAGGCCAAGGGCCAGCCTAGGGAACCCCAGGTTTACACCTTGCCTCCATCTCGGGACGAGC
TGACCAAGAACCAGGTGTCCCTGACCTGTCTCGTGAAGGGCTTCTACCCCTCCGATATCGCCGTGGA
ATGGGAGTCTAATGGCCAGCCAGAGAACAACTACAAGACAACCCCTCCTGTGCTGGACTCCGACGGC
TCATTCTTCCTGTACTCCAAGCTGACAGTGGACAAGTCCAGATGGCAGCAGGGCAACGTGTTCTCCT
GCTCCGTGATGCACGAGGCCCTGCACAATCACTACACACAGAAGTCTCTGTCTCTGAGCCCCGGCAA
GTGA
SEQ ID NO: 19: YB8 L2H5 Light chain
GAGATCGTGATGACCCAGTCTCCTGACTTCCAGAGCGTGACCCCTAAAGAGAAAGTCACCATCACCT
GTCGGGCCAGCCAGTCCATCTCTGACTACCTGCACTGGTATCAGCAGAAGCCCGATCAGTCCCCTAA
GCTGCTGATTAAGTACGCCAGCCAGAGCATCTCCGGCGTGCCATCCAGATTTTCTGGCTCCGGCTCTG
GCTCTGACTTCACCCTGACCATCAATTCCCTGGAAGCCGAGGATGCCGCCACCTACTACTGTCAGAAT
GGCCACAGCTTCCCTCTGACCTTTGGCCAGGGCACCAAGCTGGAAATCAAGAGAACCGTGGCCGCTC
CTTCCGTGTTCATCTTCCCACCATCTGACGAGCAGCTGAAGTCTGGCACCGCTTCTGTCGTGTGCCTG
CTGAACAACTTCTACCCTCGGGAAGCCAAGGTGCAGTGGAAGGTGGACAATGCCCTGCAGTCCGGC
AACTCCCAAGAGTCTGTGACCGAGCAGGACTCCAAGGACTCTACCTACAGCCTGTCCTCCACACTGAC
CCTGTCTAAGGCCGACTACGAGAAGCACAAGGTGTACGCCTGTGAAGTGACCCACCAGGGACTGTC
TAGCCCCGTGACCAAGTCTTTCAACCGGGGCGAGTGTTGA
SEQ ID NO: 20: YB8 L2H5 N297A K322A (null Fc) Heavy chain
CAGGTGCAGTTGGTGCAGTCTGGCGCCGAAGTGAAGAAACCTGGCGCTTCTGTGAAGGTGTCCTGC
AAGGCCTCTGGCTACACCTTTACCAACTACGACATCAACTGGGTCCGACAGGCTACCGGACAGGGAC
TTGAGTGGATGGGATGGATTTTCCCTGGCGACGGCAGCACCCAGTACAACGAGAAGTTTCAGGGCA
GAGTGACCATGACCACCAACACCTCCATCAGCACCGCCTACATGGAACTGTCCAGCCTGAGATCTGA
GGACACCGCCGTGTACTACTGTGCCAGACAGACCACCGCCACTTGGTTTGCTTATTGGGGCCAGGGC
ACACTGGTCACCGTGTCCTCTGCTTCTACCAAGGGACCCTCTGTGTTCCCTCTGGCTCCTTCCAGCAAG
TCTACCTCTGGTGGAACCGCTGCTCTGGGCTGCCTGGTCAAGGATTACTTTCCTGAGCCTGTGACCGT
GTCTTGGAACTCTGGTGCTCTGACCTCCGGCGTGCACACATTTCCAGCTGTGCTGCAGTCCTCCGGCC
TGTACTCTCTGTCCTCTGTCGTGACCGTGCCTTCTAGCTCTCTGGGCACCCAGACCTACATCTGCAACG
TGAACCACAAGCCTTCCAACACCAAGGTGGACAAGAAGGTGGAACCCAAGTCCTGCGACAAGACCC
ACACCTGTCCTCCATGTCCTGCTCCAGAACTGCTCGGCGGTCCAAGCGTTTTCCTGTTTCCTCCAAAGC
CTAAGGACACCCTGATGATCTCTCGGACCCCTGAAGTGACCTGCGTGGTGGTGGATGTGTCTCACGA
GGACCCAGAAGTGAAGTTCAATTGGTACGTGGACGGCGTGGAAGTGCACAACGCCAAGACCAAGCC
TAGAGAGGAACAGTACGCCTCCACCTACAGAGTGGTGTCTGTGCTGACCGTGCTGCACCAGGATTG
GCTGAACGGCAAAGAGTACAAGTGCGCCGTGTCCAACAAGGCCCTGCCTGCTCCTATCGAAAAGACC
ATCTCCAAGGCTAAGGGCCAGCCTCGGGAACCTCAGGTTTACACACTGCCTCCATCTCGGGACGAGC
TGACCAAGAATCAGGTGTCCCTGACCTGCCTCGTGAAGGGCTTCTACCCTTCCGATATCGCCGTGGA
ATGGGAGTCTAACGGCCAGCCAGAGAACAACTACAAGACAACCCCTCCTGTGCTGGACTCTGACGGC
TCATTCTTCCTGTACTCCAAGCTGACAGTGGACAAGTCTCGGTGGCAGCAGGGCAACGTGTTCTCCTG
TTCTGTGATGCACGAGGCCCTGCACAACCACTACACACAGAAGTCTCTGTCTCTGAGCCCCGGCAAG
TGA
SEQ ID NO: 21: YB8 L2H5 L234A L235A K322A (null Fc) Heavy chain
CAGGTGCAGTTGGTGCAGTCTGGCGCCGAAGTGAAGAAACCTGGCGCTTCTGTGAAGGTGTCCTGC
AAGGCCTCTGGCTACACCTTTACCAACTACGACATCAACTGGGTCCGACAGGCTACCGGACAGGGAC
TTGAGTGGATGGGATGGATTTTCCCTGGCGACGGCAGCACCCAGTACAACGAGAAGTTTCAGGGCA
GAGTGACCATGACCACCAACACCTCCATCAGCACCGCCTACATGGAACTGTCCAGCCTGAGATCTGA
GGACACCGCCGTGTACTACTGTGCCAGACAGACCACCGCCACTTGGTTTGCTTATTGGGGCCAGGGC
ACACTGGTCACCGTGTCCTCTGCTTCTACCAAGGGACCCTCTGTGTTCCCTCTGGCTCCTTCCAGCAAG
TCTACCTCTGGTGGAACCGCTGCTCTGGGCTGCCTGGTCAAGGATTACTTTCCTGAGCCTGTGACCGT
GTCTTGGAACTCTGGTGCTCTGACCTCCGGCGTGCACACATTTCCAGCTGTGCTGCAGTCCTCCGGCC
TGTACTCTCTGTCCTCTGTCGTGACCGTGCCTTCTAGCTCTCTGGGCACCCAGACCTACATCTGCAACG
TGAACCACAAGCCTTCCAACACCAAGGTGGACAAGAAGGTGGAACCCAAGTCCTGCGACAAGACCC
ACACCTGTCCTCCATGTCCTGCTCCAGAAGCTGCTGGCGGTCCAAGCGTTTTCCTGTTTCCTCCAAAGC
CTAAGGACACCCTGATGATCTCTCGGACCCCTGAAGTGACCTGCGTGGTGGTGGATGTGTCTCACGA
GGACCCAGAAGTGAAGTTCAATTGGTACGTGGACGGCGTGGAAGTGCACAACGCCAAGACCAAGCC
TAGAGAGGAACAGTACAACTCCACCTACAGAGTGGTGTCCGTGCTGACCGTGCTGCACCAGGATTG
GCTGAACGGCAAAGAGTACAAGTGCGCCGTGTCCAACAAGGCCCTGCCTGCTCCTATCGAAAAGACC
ATCTCCAAGGCTAAGGGCCAGCCTCGGGAACCTCAGGTTTACACACTGCCTCCATCTCGGGACGAGC
TGACCAAGAATCAGGTGTCCCTGACCTGCCTCGTGAAGGGCTTCTACCCTTCCGATATCGCCGTGGA
ATGGGAGTCTAACGGCCAGCCAGAGAACAACTACAAGACAACCCCTCCTGTGCTGGACTCTGACGGC
TCATTCTTCCTGTACTCCAAGCTGACAGTGGACAAGTCTCGGTGGCAGCAGGGCAACGTGTTCTCCTG
TTCTGTGATGCACGAGGCCCTGCACAACCACTACACACAGAAGTCTCTGTCTCTGAGCCCCGGCAAG
TGA
SEQ ID NO: 22: 3BH-3
CAGGTGCAGTTGGTGCAGTCTGGCGCCGAAGTGAAGAAACCTGGCGCTTCTGTGAAGGTGTCCTGC
AAGGCCTCTGGCTACACCTTTACCAACTACGACATCAACTGGGTCCGACAGGCCACCGGACAGTGTT
TGGAGTGGATGGGATGGATCTTCCCTGGCGACGGCTCTACCCAGTACAACGAGAAGTTTCAGGGCA
GAGTGACCATGACCACCAACACCTCCATCAGCACCGCCTACATGGAACTGTCCAGCCTGAGATCTGA
GGACACCGCCGTGTACTACTGTGCCAGACAGACCACCGCCACTTGGTTTGCTTATTGGGGCCAGGGC
ACACTGGTCACAGTTTCTAGCGGAGGCGGAGGAAGTGGTGGCGGAGGTTCTGGTGGCGGCGGATC
AGGCGGTGGTGGATCTGGCGGCGGTGGAAGTGGCGGAGGCGGCTCTGAAATTGTGATGACCCAGT
CTCCTGACTTCCAGAGCGTGACCCCTAAAGAGAAAGTCACCATCACCTGTCGGGCCAGCCAGTCCAT
CTCTGACTACCTGCACTGGTATCAGCAGAAGCCCGATCAGTCCCCTAAGCTGCTGATTAAGTACGCCA
GCCAGAGCATCTCCGGCGTGCCATCCAGATTTTCTGGCTCCGGCTCTGGCTCTGACTTCACCCTGACC
ATCAATTCCCTGGAAGCCGAGGATGCCGCCACCTACTACTGTCAGAATGGCCACAGCTTCCCTCTGAC
CTTCGGCTGTGGCACCAAGCTGGAAATCAAAGGCGGCGGAGGCTCAGGCGGAGGTGGAAGCGGAG
GTGGCGGTTCCGGCGGTGGCGGAAGTCATGTTCAACTGGTTGAATCCGGCGGAGGATTGGTGCAGC
CAGGCGGATCTCTGAGACTGTCTTGTGCCGCTTCCGGCTTCTCCCTGACTGATTATGGCGTGCACTGG
GTTCGACAAGCCCCTGGCAAAGGACTGGAATGGCTGGGAGTTATTTGGAGCGGCGGAGGAACCGC
CTACAACACCGCTCTGATCTCCCGGTTCACCATCAGCCGGGACAACTCCAAGAACACCCTGTACCTGC
AGATGAACTCTCTGAGAGCCGAAGATACCGCTGTGTATTACTGCGCTCGGAGAGGCAGCTACCCCTA
CAACTACTTTGATGCTTGGGGCTGCGGAACCCTGGTTACAGTCTCTTCTGGCGGCGGAGGCAGCGGA
GGTGGTGGTTCTGGCGGAGGCGGATCTGGTGGCGGAGGTAGTGGCGGCGGTGGAAGCGGTGGCG
GAGGATCTCAAGCTGTGGTCACACAAGAGCCCAGCCTGACAGTTTCTCCTGGCGGAACCGTTACACT
GACCTGTGGATCTTCTACCGGCGCTGTGACCGCCTCTAACTACGCTAACTGGGTGCAGCAGAAACCC
GGCCAGTGTCCTAGAGGCCTGATCGGCGGACACAACAATAGACCTCCAGGCGTGCCCGCTAGATTCT
CTGGATCTCTGCTTGGCGGCAAGGCTGCTCTGACACTTTTGGGCGCTCAGCCTGAGGATGAGGCTGA
GTACTATTGCGCCCTGTGGTACTCCGACCATTGGGTTATCGGCGGAGGGACCAAACTGACCGTTCTG
GGAACACCTCTGGGCGACACCACACATACCTCTGGAAAGCCTCTGGACGGCGAGTACTTCACACTGC
AGATCCGGGGCAGAGAACGCTTCGAGATGTTCAGAGAGCTGAACGAGGCCCTGGAACTGAAGGAT
GCCCAGGCTGGAAAAGAACCTGGTGGTTCAGGTGGCGCCCCTCACCACCATCATCACCATTAAGGAT
CCAAGG
SEQ ID NO: 23: Murine 8H9 Heavy chain
QVQLQQSGAELVKPGASVKLSCKASGYTFTNYDINWVRQRPEQGLEWIGWIFPGDGSTQYNEKFKGKA
TLTTDTSSSTAYMQLSRLTSEDSAVYFCARQTTATWFAYWGQGTLVTVSAAKTTPPSVYPLAPGSAAQTN
SMVTLGCLVKGYFPEPVTVTWNSGSLSSGVHTFPAVLQSDLYTLSSSVTVPSSTWPSETVTCNVAHPASST
KVDKKIVPRDCGCKPCICTVPEVSSVFIFPPKPKDVLTITLTPKVTCVVVDISKDDPEVQFSWFVDDVEVHT
AQTQPREEQFNSTFRSVSELPIMHQDWLNGKEFKCRVNSAAFPAPIEKTISKTKGRPKAPQVYTIPPPKEQ
MAKDKVSLTCMITDFFPEDITVEWQWNGQPAENYKNTQPIMDTDGSYFVYSKLNVQKSNWEAGNTFT
CSVLHEGLHNHHTEKSLSHSPGK
SEQ ID NO: 24: Murine 8H9 Light Chain
DIVMTQSPATLSVTPGDRVSLSCRASQSISDYLHWYQQKSHESPRLLIKYASQSISGIPSRFSGSGSGSDFTL
SINSVEPEDVGVYYCQNGHSFPLTFGAGTKELKRADAAPTVSIFPPSSEQLTSGGASVVCFLNNFYPDKIN
VKWKIDGSERQNGVLNSWTDQDSKDSTYSMSSTLTLTKDEYERHNSYTCEATHKTSTSPIVKSFNRNEC
SEQ ID NO: 25: 8H9 Heavy Chain CDR-1
NYDIN
SEQ ID NO: 26: 8H9 Heavy Chain CDR-2
WIFPGDGSTQYNEKFKG
SEQ ID NO: 27: 8H9 Heavy Chain CDR-3
QTTATWFAY
SEQ ID NO: 28: 8H9 Light Chain CDR-1
RASQSISDYLH
SEQ ID NO: 29: 8H9 Light Chain CDR-2
YASQSIS
SEQ ID NO: 30: 8H9 Light Chain CDR-3
QNGHSFPLT
SEQ ID NO: 31: 4Ig-B7H3
MLRRRGSPGMGVHVGAALGALWFCLTGALEVQVPEDPVVALVGTDATLCCSFSPEPGFSLAQLNLIWQ
LTDTKQLVHSFAEGQDQGSAYANRTALFPDLLAQGNASLRLQRVRVADEGSFTCFVSIRDFGSAAVSLQV
AAPYSKPSMTLEPNKDLRPGDTVTITCSSYQGYPEAEVFWQDGQGVPLTGNVTTSQMANEQGLFDVHS
ILRVVLGANGTYSCLVRNPVLQQDAHSSVTITPQRSPTGAVEVQVPEDPVVALVGTDATLRCSFSPEPGFS
LAQLNLIWQLTDTKQLVHSFTEGRDQGSAYANRTALFPDLLAQGNASLRLQRVRVADEGSFTCFVSIRDF
GSAAVSLQVAAPYSKPSMTLEPNKDLRPGDTVTITCSSYRGYPEAEVFWQDGQGVPLTGNVTTSQMAN
EQGLFDVHSVLRVVLGANGTYSCLVRNPVLQQDAHGSVTITGQPMTFPPEALWVTVGLSVCLIALLVALA
FVCWRKIKQSCEEENAGAEDQDGEGEGSKTALQPLKHSDSKEDDGQEIA
SEQ ID NO: 32: 2Ig-B7H3
MLRRRGSPGMGVHVGAALGALWFCLTGALEVQVPEDPVVALVGTDATLCCSFSPEPGFSLAQLNLIWQ
LTDTKQLVHSFAEGQDQGSAYANRTALFPDLLAQGNASLRLQRVRVADEGSFTCFVSIRDFGSAAVSLQV
AAPYSKPSMTLEPNKDLRPGDTVTITCSSYRGYPEAEVFWQDGQGVPLTGNVTTSQMANEQGLFDVHS
VLRVVLGANGTYSCLVRNPVLQQDAHGSVTITGQPMTFPPEALWVTVGLSVCLIALLVALAFVCWRKIKQ
SCEEENAGAEDQDGEGEGSKTALQPLKHSDSKEDDGQEIA
SEQ ID NO: 33: B7H3 Epitope
IRFD
SEQ ID NO: 34: Antibody ch8H9 Fab Heavy chain
QVQLQQSGAELVKPGASVKLSCKASGYTFTNYDINWVRQRPEQGLEWIGWIFPGDGSTQYNEKFKGKA
TLTTDTSSSTAYMQLSRLTSEDSAVYFCARQTTATWFAYWGQGTLVTVSAASTKGPSVFPLAPSSKSTSG
GTAALGCLVKDYFPEPVTVSWNSGALTSGVHTFPAVLQSSGLYSLSSVVTVPSSSLGTQTYICNVNHKPSN
TKVDKRVEPKS
SEQ ID NO: 35: Antibody ch8H9 Fab Light chain
DIVMTQSPATLSVTPGDRVSLSCRASQSISDYLHWYQQKSHESPRLLIKYASQSISGIPSRFSGSGSGSDFTL
SINSVEPEDVGVYYCQNGHSFPLTFGAGTKLELKRTVAAPSVFIFPPSDEQLKSGTASVVCLLNNFYPREAK
VQWKVDNALQSGNSQESVTEQDSKDSTYSLSSTLTLSKADYEKHKVYACEVTHQGLSSPVTKSFNRGE
SEQ ID NO: 36: IGKV3D-11
EIVLTQSPATLSLSPGERATLSCRASQSVSSYLAWYQQKPGQAPRLLIYDASNRATGIPARFSGSGPGTDFT
LTISSLEPEDFAVYYCQQRSNWH
SEQ ID NO: 37: IGKV6-21
EIVLTQSPDFQSVTPKEKVTITCRASQSIGSSLHWYQQKPDQSPKLLIKYASQSFSGVPSRFSGSGSGTDFTL
TINSLEAEDAATYYCHQSSSLP
SEQ ID NO: 38: IGHV1-8
QVQLVQSGAEVKKPGASVKVSCKASGYTFTSYDINWVRQATGQGLEWMGWMNPNSGNTGYAQKFQ
GRVTMTRNTSISTAYMELSSLRSEDTAVYYCAR
SEQ ID NO: 39: IGHV1-46
QVQLVQSGAEVKKPGASVKVSCKASGYTFTSYYMHWVRQAPGQGLEWMGIINPSGGSTSYAQKFQGR
VTMTRDTSTSTVYMELSSLRSEDTAVYYCAR
SEQ ID NO: 40: LFR1 VL L2H5
EIVMTQSPDFQSVTPKEKVTITC
SEQ ID NO: 41: LFR2 VL L2H5
WYQQKPDQSPKLLIK
SEQ ID NO: 42: LFR3 VL L2H5
GVPSRFSGSGSGSDFTLTINSLEAEDAATYYC
SEQ ID NO: 43: LFR4 VL L2H5
FGQGTKLEIK
SEQ ID NO: 44: HFR1 VH L2H5
QVQLVQSGAEVKKPGASVKVSCKASGYTFT
SEQ ID NO: 45: HFR2 VH L2H5
WVRQATGQGLEWMG
SEQ ID NO: 46: HFR3 VH L2H5
RVTMTTNTSISTAYMELSSLRSEDTAVYYCAR
SEQ ID NO: 47: HFR4 VH L2H5
WGQGTLVTVSS
SEQ ID NO: 48: LFR1 VL L2H3
EIVMTQSPDFQSVTPKEKVTITC
SEQ ID NO: 49: LFR2 VL L2H3
WYQQKPDQSPKLLIK
SEQ ID NO: 50: LFR3 VL L2H3
GVPSRFSGSGSGSDFTLTINSLEAEDAATYYC
SEQ ID NO: 51: LFR4 VL L2H3
FGQGTKLEIK
SEQ ID NO: 52: HER1 VH L2H3
QVQLVQSGAEVKKPGASVKVSCKASGYTFT
SEQ ID NO: 53: HFR2 VH L2H3
WVRQATGQGLEWMG
SEQ ID NO: 54: HFR3 VH L2H3
RVTLTRNTSISTAYMELSSLRSEDTAVYFCAR
SEQ ID NO: 55: HFR4 VH L2H3
WGQGTLVTVSS
SEQ ID NO: 56: LFR1 VL L2H4
EIVMTQSPDFQSVTPKEKVTITC
SEQ ID NO: 57: LFR2 VL L2H4
WYQQKPDQSPKLLIK
SEQ ID NO: 58: LFR3 VL L2H4
GVPSRFSGSGSGSDFTLTINSLEAEDAATYYC
SEQ ID NO: 59: LFR4 VL L2H4
FGQGTKLEIK
SEQ ID NO: 60: HFR1 VH L2H4
QVQLVQSGAEVKKPGASVKVSCKASGYTFT
SEQ ID NO: 61: HFR2 VH L2H4
WVRQATGQGLEWIG
SEQ ID NO: 62: HFR3 VH L2H4
RVTMTTNTSISTAYMELSSLRSEDTAVYFCAR
SEQ ID NO: 63: HFR4 VH L2H4
WGQGTLVTVSS
SEQ ID NO: 64: IMGT CDR murine 8H9 heavy chain CDR1
GYTFTNYD
SEQ ID NO: 65: IMGT CDR murine 8H9 heavy chain CDR2
IFPGDGST
SEQ ID NO: 66: IMGT CDR murine 8H9 heavy chain CDR3
ARQTTATWFAY
SEQ ID NO: 67: IMGT CDR murine 8H9 light chain CDR1
QSISDY
SEQ ID NO: 68: IMGT CDR murine 8H9 light chain CDR2
YA
SEQ ID NO: 69: IMGT CDR murine 8H9 light chain CDR3
QNGHSFPLT
SEQ ID NO: 70: IMGT CDR H1 heavy chain CDR1
GYTFTNYD
SEQ ID NO: 71: IMGT CDR H1 heavy chain CDR2
IFPGDGST
SEQ ID NO: 72: IMGT CDR H1 heavy chain CDR3
ARQTTATWFAY
SEQ ID NO: 73: IMGT CDR L1 light chain CDR1
QSISDY
SEQ ID NO: 74: IMGT CDR L1 light chain CDR2
YA
SEQ ID NO: 75: IMGT CDR L1 light chain CDR3
QNGHSFPLT
SEQ ID NO: 76: IMGT CDR H2 heavy chain CDR1
GYTFTNYD
SEQ ID NO: 77: IMGT CDR H2 heavy chain CDR2
IFPGDGST
SEQ ID NO: 78: IMGT CDR H2 heavy chain CDR3
ARQTTATWFAY
SEQ ID NO: 79: IMGT CDR L2 light chain CDR1
QSISDY
SEQ ID NO: 80: IMGT CDR L2 light chain CDR2
YA
SEQ ID NO: 81: IMGT CDR L2 light chain CDR3
QNGHSFPLT
SEQ ID NO: 82: IMGT CDR H3 heavy chain CDR1
GYTFTNYD
SEQ ID NO: 83: IMGT CDR H3 heavy chain CDR2
IFPGDGST
SEQ ID NO: 84: IMGT CDR H3 heavy chain CDR3
ARQTTATWFAY
SEQ ID NO: 85: IMGT CDR L3 light chain CDR1
QSISDY
SEQ ID NO: 86: IMGT CDR L3 light chain CDR2
YA
SEQ ID NO: 87: IMGT CDR L3 light chain CDR3
QNGHSFPLT
SEQ ID NO: 88: IMGT CDR H4 heavy chain CDR1
GYTFTNYD
SEQ ID NO: 89: IMGT CDR H4 heavy chain CDR2
IFPGDGST
SEQ ID NO: 90: IMGT CDR H4 heavy chain CDR3
ARQTTATWFAY
SEQ ID NO: 91: IMGT CDR L4 light chain CDR1
QSISDY
SEQ ID NO: 92: IMGT CDR L4 light chain CDR2
YA
SEQ ID NO: 93: IMGT CDR L4 light chain CDR3
QNGHSFPLT
SEQ ID NO: 94: IMGT CDR H5 heavy chain CDR1
GYTFTNYD
SEQ ID NO: 95: IMGT CDR H5 heavy chain CDR2
IFPGDGST
SEQ ID NO: 96: IMGT CDR H5 heavy chain CDR3
ARQTTATWFAY
SEQ ID NO: 97: IMGT CDR H6 heavy chain CDR1
GYTFTNYD
SEQ ID NO: 98: IMGT CDR H6 heavy chain CDR2
IFPGDGST
SEQ ID NO: 99: IMGT CDR H6 heavy chain CDR3
ARQTTATWFAY
SEQ ID NO: 100: m8H9 LFR1
DIVMTQSPATLSVTPGDRVSLSC
SEQ ID NO: 101: m8H9 LFR2
WYQQKSHESPRLLIK
SEQ ID NO: 102: m8H9 LFR3
GIPSRFSGSGSGSDFTLSINSVEPEDVGVYYC
SEQ ID NO: 103: m8H9 LFR4
FGAGTKLELK
SEQ ID NO: 104: m8H9 HFR1
QVQLQQSGAELVKPGASVKLSCKASGYTFT
SEQ ID NO: 105: m8H9 HFR2
WVRQRPEQGLEWIG
SEQ ID NO: 106: m8H9 HFR3
KATLTTDTSSSTAYMQLSRLTSEDSAVYFCAR
SEQ ID NO: 107: m8H9 HFR4
WGQGTLVTVSA
SEQ ID NO: 108: L1 LFR1
EIVLTQSPDFQSVTPKEKVTLTC
SEQ ID NO: 109: L1 LFR2
WYQQKPDQSPKLLIK
SEQ ID NO: 110: L1 LFR3
GVPSRFSGSGSGSDFTLTINSLEAEDAATYYC
SEQ ID NO: 111: L1 LFR4
FGQGTKLEIK
SEQ ID NO: 112: L3 LFR1
EIVMTQSPDFQSVTPKEKVTLTC
SEQ ID NO: 113: L3 LFR2
WYQQKPDQSPKLLIK
SEQ ID NO: 114: L3 LFR3
GVPSRFSGSGSGSDFTLTINSLEAEDAGVYYC
SEQ ID NO: 115: L3 LFR4
FGQGTKLEIK
SEQ ID NO: 116: L4 LFR1
EIVMTQSPDFQSVTPKEKVTLTC
SEQ ID NO: 117: L4 LFR2
WYQQKPDQSPKLLIK
SEQ ID NO: 118: L4 LFR3
GIPSRFSGSGSGTDFTLTINSVEAEDAGVYYC
SEQ ID NO: 119: L4 LFR4
FGQGTKLEIK
SEQ ID NO: 120: H1 HFR1
QVQLVQSGAEVKKPGASVKVSCKASGYTFT
SEQ ID NO: 121: H1 HFR2
WVRQATGQGLEWIG
SEQ ID NO: 122: H1 HFR3
RVTLTTNTSISTAYMELSSLRSEDTAVYYCAR
SEQ ID NO: 123: H1 HFR4
WGQGTLVTVSS
SEQ ID NO: 124: H2 HFR1
QVQLQQSGAEVKKPGASVKVSCKASGYTFT
SEQ ID NO: 125: H2 HFR2
WVRQATGQGLEWIG
SEQ ID NO: 126: H2 HFR3
RATLTRNTSISTAYMELSSLRSEDTAVYYCAR
SEQ ID NO: 127: H2 HFR4
WGQGTLVTVSS
SEQ ID NO: 128: H6 HFR1
QVQLQQSGAEVKKPGASVKLSCKASGYTFT
SEQ ID NO: 129: H6 HFR2
WVRQATGQGLEWIG
SEQ ID NO: 130: H6 HFR3
RATLTTNTSISTAYMELSSLRSEDTAVYFCAR
SEQ ID NO: 131: H6 HFR4
WGQGTLVTVSS
SEQ ID NO: 132: 3BH-3 without His tag
QVQLVQSGAEVKKPGASVKVSCKASGYTFTNYDINWVRQATGQCLEWMGWIFPGDGSTQYNEKFQG
RVTMTTNTSISTAYMELSSLRSEDTAVYYCARQTTATWFAYWGQGTLVTVSSGGGGSGGGGSGGGGSG
GGGSGGGGSGGGGSEIVMTQSPDFQSVTPKEKVTITCRASQSISDYLHWYQQKPDQSPKLLIKYASQSIS
GVPSRFSGSGSGSDFTLTINSLEAEDAATYYCQNGHSFPLTFGCGTKLEIKGGGGSGGGGSGGGGSGGG
GSHVQLVESGGGLVQPGGSLRLSCAASGFSLTDYGVHWVRQAPGKGLEWLGVIWSGGGTAYNTALISR
FTISRDNSKNTLYLQMNSLRAEDTAVYYCARRGSYPYNYFDAWGCGTLVTVSSGGGGSGGGGSGGGGS
GGGGSGGGGSGGGGSQAVVTQEPSLTVSPGGTVTLTCGSSTGAVTASNYANWVQQKPGQCPRGLIGG
HNNRPPGVPARFSGSLLGGKAALTLLGAQPEDEAEYYCALWYSDHWVIGGGTKLTVLGTPLGDTTHTSG
KPLDGEYFTLQIRGRERFEMFRELNEALELKDAQAGKEPGGSGGAP
SEQ ID NO: 133: LFR4 VL L2H5 alternative
FGCGTKLEIK
SEQ ID NO: 134: HFR2 VH L2H5 alternative
WVRQATGQCLEWMG
SEQ ID NO: 135: YB8 H5 alternative
QVQLVQSGAEVKKPGASVKVSCKASGYTFTNYDINWVRQATGQCLEWMGWIFPGDGSTQYNEKFQG
RVTMTTNTSISTAYMELSSLRSEDTAVYYCARQTTATWFAYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGK
SEQ ID NO: 136: YB8 H5 N297A and K322A alternative
QVQLVQSGAEVKKPGASVKVSCKASGYTFTNYDINWVRQATGQCLEWMGWIFPGDGSTQYNEKFQG
RVTMTTNTSISTAYMELSSLRSEDTAVYYCARQTTATWFAYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRTPEVTCVVVDVSHEDPEVKFNW
YVDGVEVHNAKTKPREEQYASTYRVVSVLTVLHQDWLNGKEYKCAVSNKALPAPIEKTISKAKGQPREPQ
VYTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGK
SEQ ID NO: 137: YB8_H5 L234A, L235A and K322A alternative
QVQLVQSGAEVKKPGASVKVSCKASGYTFTNYDINWVRQATGQCLEWMGWIFPGDGSTQYNEKFQG
RVTMTTNTSISTAYMELSSLRSEDTAVYYCARQTTATWFAYWGQGTLVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKPS
NTKVDKKVEPKSCDKTHTCPPCPAPEAAGGPSVFLFPPKPKDTLMISRTPEVTCVVVDVSHEDPEVKFNW
YVDGVEVHNAKTKPREEQYNSTYRVVSVLTVLHQDWLNGKEYKCAVSNKALPAPIEKTISKAKGQPREPQ
VYTLPPSRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGK
SEQ ID NO: 138: YB8 L2 alternative
EIVMTQSPDFQSVTPKEKVTITCRASQSISDYLHWYQQKPDQSPKLLIKYASQSISGVPSRFSGSGSGSDFT
LTINSLEAEDAATYYCQNGHSFPLTFGCGTKLEIKRTVAAPSVFIFPPSDEQLKSGTASVVCLLNNFYPREAK
VQWKVDNALQSGNSQESVTEQDSKDSTYSLSSTLTLSKADYEKHKVYACEVTHQGLSSPVTKSFNRGEC
SEQ ID NO: 139: Tetramerization domain, SADA polypeptide
KPLDGEYFTLQIRGRERFEMFRELNEALELKDAQAGKEP

The content of the ASCII text file of the sequence listing named “Substitute-Sequence-Listing-31Mar2023-12397-2201”, having a size of 131 kb and a creation date of 30 Mar. 2023, and electronically submitted via EFS-Web on 31 Mar. 2023, is incorporated herein by reference in its entirety.

### REFERENCES

1. Chapoval, A. I., et al., B7-H3: A costimulatory molecule for T cell activation and IFN-γ production. Nature Immunology, 2001. 2(3): p. 269-274.
2. Steinberger, P., et al., Molecular Characterization of Human 4Ig-B7-H3, a Member of the 87 Family with Four Ig-Like Domains. The Journal of Immunology, 2004. 172(4): p. 2352-2359.
3. Loos, M., et al., B7-H3 and Its Role in Antitumor Immunity. Clinical and Developmental Immunology, 2010. 2010: p. 1-7.
4. Modak, S., et al., Monoclonal antibody 8H9 targets a novel cell surface antigen expressed by a wide spectrum of human solid tumors. Cancer Res, 2001. 61(10): p. 4048-54.
5. Roth, T. J., et al., B7-H3 ligand expression by prostate cancer: a novel marker of prognosis and potential target for therapy. Cancer Res, 2007. 67(16): p. 7893-900.
6. Zang, X., et al., B7-H3 and 87x are highly expressed in human prostate cancer and associated with disease spread and poor outcome. Proc Natl Acad Sci USA, 2007. 104(49): p. 19458-63.
7. Crispen, P. L., et al., Tumor cell and tumor vasculature expression of B7-H3 predict survival in clear cell renal cell carcinoma. Clin Cancer Res, 2008. 14(16): p. 5150-7.
8. Zang, X., et al., Tumor associated endothelial expression of B7-H3 predicts survival in ovarian carcinomas. Modern Pathology, 2010. 23(8): p. 1104-1112.
9. Lemke, D., et al., Costimulatory Protein 4IgB7H3 Drives the Malignant Phenotype of Glioblastoma by Mediating Immune Escape and Invasiveness. Clinical Cancer Research, 2012. 18(1): p. 105-117.
10. Wang, L., et al., B7-H3 is overexpressed in patients suffering osteosarcoma and associated with tumor aggressiveness and metastasis. PLoS One, 2013.8(8): p. e70689.
11. Gregorio, A., et al., Small round blue cell tumours: diagnostic and prognostic usefulness of the expression of B7-H3 surface molecule. Histopathology, 2008. 53(1): p. 73-80.
12. Du, H., et al., Antitumor Responses in the Absence of Toxicity in Solid Tumors by Targeting B7-H3 via Chimeric Antigen Receptor T Cells. Cancer Cell, 2019. 35(2): p. 221-237 e8.
13. Majzner, R. G., et al., CAR T Cells Targeting B7-H3, a Pan-Cancer Antigen, Demonstrate Potent Preclinical Activity Against Pediatric Solid Tumors and Brain Tumors. Clin Cancer Res, 2019. 25(8): p. 2560-2574.
14. Castellanos, J. R., et al., B7-H3 role in the immune landscape of cancer. American Journal of Clinical and Experimental Immunology, 2017. 6(4): p. 66-75.
15. Kramer, K., et al., Compartmental intrathecal radioimmunotherapy: results for treatment for metastatic CNS neuroblastoma. J Neurooncol, 2010. 97(3): p. 409-18.
16. Van Cutsem, E., et al., Gastric cancer. Lancet, 2016. 388(10060): p. 2654-2664.
17. Ahmed, M., et al., Humanized Affinity-matured Monoclonal Antibody 8H9 Has Potent Antitumor Activity and Binds to FG Loop of Tumor Antigen B7-H3. Journal of Biological Chemistry, 2015. 290(50): p. 30018-30029.
18. Siegel, R L. et al. Cancer statistics, 2020. CA: A Cancer Journal for Clinicians, Vol 70, Issue 1, January/February 2020, p. 7-30
19. American Cancer Society website, survival rates www.cancer.org/cancer/ovarian-cancer/detection-diagnosis-staging/survival-rates.html, Accessed May 26, 2020

1.-86. (canceled)

87. A humanized antibody or antigen binding fragment thereof, capable of binding to B7H3 antigen, comprising
CDR regions having a total of at least 90% identity to the CDR sequences selected from the group consisting of SEQ ID NOS: 25-30 and 64-99, and
FR regions having a total of at least 70% identity to the FR sequences selected from the group consisting of SEQ ID NOS: 40-63, 108-131, 133 and 134.

88. The humanized antibody or antibody binding fragment thereof of claim 87, wherein the CDR regions are selected from the group consisting of SEQ ID NOS: 25-30 and 64-99.

89. The humanized antibody or antibody binding fragment of claim 88, wherein the heavy chain CDR sequences are the sequences of SEQ ID NOS:64-66 and the light chain CDR sequences are the sequences of SEQ ID NOS: 67-69.

90. The humanized antibody or antibody binding fragment of claim 87, comprising FR regions selected from the group consisting of SEQ ID NOS: 40-47, and sequences of at least 90% sequence identity to these sequences.

91. The humanized antibody or antibody binding fragment of claim 90, comprising heavy chain FR regions of SEQ ID NOS: 40-43 and light chain FR regions of SEQ ID NOS: 44-47.

92. The humanized antibody or antibody binding fragment of claim 87, comprising a heavy chain variable region VH selected from the group consisting of SEQ ID NOS: 2-9.

93. The humanized antibody or antibody binding fragment of claim 87, comprising light chain variable region VL selected from the group consisting of SEQ ID NOS: 11-14.

94. The humanized antibody or antibody binding fragment of claim 87, comprising a heavy chain variable region VH of SEQ ID NO: 6, and a light chain variable region VL of SEQ ID NO: 12.

95. A self-assembly disassembly (SADA) polypeptide, wherein said polypeptide is linked to an antibody or antigen binding fragment according to claim 87.

96. The self-assembly disassembly (SADA) polypeptide of claim 95, wherein said antibody or antigen binding fragment thereof is a bispecific and/or trispecific binding antibody.

97. The self-assembly disassembly (SADA) polypeptide of claim 96, wherein the bispecific and/or trispecific binding antibody comprises a first antibody or antigen binding fragment according to claim 87, and a second antibody or antigen binding fragment thereof capable of binding DOTA.

98. The self-assembly disassembly (SADA) polypeptide of claim 97, comprising a sequence selected from the group consisting of SEQ ID NO: 16, SEQ ID NO: 17 and SEQ ID NO: 132.

99. An isolated nucleic acid encoding a humanized antibody or antigen binding fragment according to claim 87 or encoding a self-assembly disassembly (SADA) polypeptide linked to an antibody or antigen binding fragment according to claim 87.

100. A recombinant vector comprising the isolated nucleic acid molecule of claim 99.

101. A host cell comprising the isolated nucleic acid of claim 99 or a recombinant vector comprising the isolated nucleic acid molecule according to claim 99.

102. A pharmaceutical composition comprising the antibody or antigen binding fragment of claim 87 or a self-assembly disassembly (SADA) polypeptide linked to an antibody or antigen binding fragment according to claim 87.

103. A method of treating, preventing, alleviating and/or diagnosing symptoms of a medical condition in a subject, comprising a step of administering the antibody or antigen binding fragment of claim 87, a self-assembly disassembly (SADA) polypeptide linked to an antibody or antigen binding fragment according to claim 87, or a pharmaceutical composition comprising said antibody or antigen binding fragment thereof or comprising a SADA polypeptide linked to an antibody or antigen binding fragment according to claim 87, wherein said medical condition is characterized by expression of B7H3 antigen.

104. The method of claim 103, wherein the medical condition is a cancer, such as a cancer tumor or metastasis; wherein said tumor and/or said metastasis is prostate cancer, a desmoplastic small round cell tumor, ovarian cancer, gastric cancer, pancreatic cancer, liver cancer, renal cancer, breast cancer, non-small cell lung cancer, melanoma, alveolar rhabdomyosarcoma, embryonal rhabdomyosarcoma, Ewing sarcoma, Wilms tumor, neuroblastoma, ganglioneuroblastoma, ganglioneuroma, medulloblastoma, high-grade glioma, diffuse intrinsic pontine glioma, embryonal tumors with multilayered rosettes, or a cancer expressing B7H3.


SEQUENCE LISTING (139 sequences)

SEQ ID NO: 1 - protein, 448 aa, Artificial Sequence
  Chimeric Heavy Chain (Parental)
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 448 aa, Artificial Sequence
  YB8 H1
QVQLVQSGAEVKKPGASVKVSCKASGYTFTNYDINWVRQATGQGLEWIGWIFPGDGSTQYNEKFQGRVTLTTNTSISTAYMELSSLRSEDTAVYYCARQTTATWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 448 aa, Artificial Sequence
  YB8 H2
QVQLQQSGAEVKKPGASVKVSCKASGYTFTNYDINWVRQATGQGLEWIGWIFPGDGSTQYNEKFKGRATLTRNTSISTAYMELSSLRSEDTAVYYCARQTTATWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 448 aa, Artificial Sequence
  YB8 H3
QVQLVQSGAEVKKPGASVKVSCKASGYTFTNYDINWVRQATGQGLEWMGWIFPGDGSTQYNEKFKGRVTLTRNTSISTAYMELSSLRSEDTAVYFCARQTTATWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 448 aa, Artificial Sequence
  YB8 H4
QVQLVQSGAEVKKPGASVKVSCKASGYTFTNYDINWVRQATGQGLEWIGWIFPGDGSTQYNEKFQGRVTMTTNTSISTAYMELSSLRSEDTAVYFCARQTTATWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 448 aa, Artificial Sequence
  YB8 H5
QVQLVQSGAEVKKPGASVKVSCKASGYTFTNYDINWVRQATGQGLEWMGWIFPGDGSTQYNEKFQGRVTMTTNTSISTAYMELSSLRSEDTAVYYCARQTTATWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 448 aa, Artificial Sequence
  YB8 H5 N297A and K322A
QVQLVQSGAEVKKPGASVKVSCKASGYTFTNYDINWVRQATGQGLEWMGWIFPGDGSTQYNEKFQGRVTMTTNTSISTAYMELSSLRSEDTAVYYCARQTTATWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 448 aa, Artificial Sequence
  YB8 H5 L234A L235A and K322A
QVQLVQSGAEVKKPGASVKVSCKASGYTFTNYDINWVRQATGQGLEWMGWIFPGDGSTQYNEKFQGRVTMTTNTSISTAYMELSSLRSEDTAVYYCARQTTATWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 448 aa, Artificial Sequence
  YB8 H6
QVQLQQSGAEVKKPGASVKLSCKASGYTFTNYDINWVRQATGQGLEWIGWIFPGDGSTQYAQKFQGRATLTTNTSISTAYMELSSLRSEDTAVYFCARQTTATWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial Sequence
  Chimeric Light chain (parental)
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 214 aa, Artificial Sequence
  YB8 L1
EIVLTQSPDFQSVTPKEKVTLTCRASQSISDYLHWYQQKPDQSPKLLIKYASQSISGVPSRFSGSGSGSDFTLTINSLEAEDAATYYCQNGHSFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 214 aa, Artificial Sequence
  YB8 L2
EIVMTQSPDFQSVTPKEKVTITCRASQSISDYLHWYQQKPDQSPKLLIKYASQSISGVPSRFSGSGSGSDFTLTINSLEAEDAATYYCQNGHSFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 214 aa, Artificial Sequence
  YB8 L3
EIVMTQSPDFQSVTPKEKVTLTCRASQSISDYLHWYQQKPDQSPKLLIKYASQSISGVPSRFSGSGSGSDFTLTINSLEAEDAGVYYCQNGHSFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 214 aa, Artificial Sequence
  YB8 L4
EIVMTQSPDFQSVTPKEKVTLTCRASQSISDYLHWYQQKPDQSPKLLIKYASQSISGIPSRFSGSGSGTDFTLTINSVEAEDAGVYYCQNGHSFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 597 aa, Artificial Sequence
  3BH-1
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAGGGGSGGGGSGGGGSGGGGSGGGGSGGGGSDIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGAPHHHHHH

SEQ ID NO: 16 - protein, 597 aa, Artificial Sequence
  3BH-2
QVQLVQSGAEVKKPGASVKVSCKASGYTFTNYDINWVRQATGQGLEWMGWIFPGDGSTQYNEKFQGRVTMTTNTSISTAYMELSSLRSEDTAVYYCARQTTATWFAYWGQGTLVTVSSGGGGSGGGGSGGGGSGGGGSGGGGSGGGGSEIVMTQSPDFQSVTPKEKVTITCRASQSISDYLHWYQQKPDQSPKLLIKYASQSISGVPSRFSGSGSGSDFTLTINSLEAEDAATYYCQNGHSFPLTFGQGTKLEIKGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGAPHHHHHH

SEQ ID NO: 17 - protein, 597 aa, Artificial Sequence
  3BH-3
QVQLVQSGAEVKKPGASVKVSCKASGYTFTNYDINWVRQATGQCLEWMGWIFPGDGSTQYNEKFQGRVTMTTNTSISTAYMELSSLRSEDTAVYYCARQTTATWFAYWGQGTLVTVSSGGGGSGGGGSGGGGSGGGGSGGGGSGGGGSEIVMTQSPDFQSVTPKEKVTITCRASQSISDYLHWYQQKPDQSPKLLIKYASQSISGVPSRFSGSGSGSDFTLTINSLEAEDAATYYCQNGHSFPLTFGCGTKLEIKGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGAPHHHHHH

SEQ ID NO: 18 - DNA, 1347 bp, Artificial Sequence
  YB8 L2H5 heavy chain
caggttcagttgcagcagtctggcgccgaagtgaagaaacctggcgcctccgtgaagctgtcctgcaaggcttctggctacaccttcaccaactacgacatcaactgggtccgacaggctaccggacagggactcgaatggatcggctggatctttcctggcgacggctctacccagtacgcccagaaatttcaggggagagctaccctgaccaccaacacctctatctccaccgcctacatggaactgtccagcctgagatccgaggataccgccgtgtacttctgtgccagacagaccaccgccacttggtttgcttattggggccagggcaccctggtcaccgtttcttccgcttctaccaagggacccagcgtgttccctctggctccttccagcaagtctacctctggcggaacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgtctcgtgaagggcttctacccctccgatatcgccgtggaatgggagtctaatggccagccagagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacacagaagtctctgtctctgagccccggcaagtga

SEQ ID NO: 19 - DNA, 645 bp, Artificial Sequence
  YB8 L2H5 light chain
gagatcgtgatgacccagtctcctgacttccagagcgtgacccctaaagagaaagtcaccatcacctgtcgggccagccagtccatctctgactacctgcactggtatcagcagaagcccgatcagtcccctaagctgctgattaagtacgccagccagagcatctccggcgtgccatccagattttctggctccggctctggctctgacttcaccctgaccatcaattccctggaagccgaggatgccgccacctactactgtcagaatggccacagcttccctctgacctttggccagggcaccaagctggaaatcaagagaaccgtggccgctccttccgtgttcatcttcccaccatctgacgagcagctgaagtctggcaccgcttctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaaccggggcgagtgttga

SEQ ID NO: 20 - DNA, 1347 bp, Artificial Sequence
  YB8 L2H5 N297A K322A null Fc heavy chain
caggtgcagttggtgcagtctggcgccgaagtgaagaaacctggcgcttctgtgaaggtgtcctgcaaggcctctggctacacctttaccaactacgacatcaactgggtccgacaggctaccggacagggacttgagtggatgggatggattttccctggcgacggcagcacccagtacaacgagaagtttcagggcagagtgaccatgaccaccaacacctccatcagcaccgcctacatggaactgtccagcctgagatctgaggacaccgccgtgtactactgtgccagacagaccaccgccacttggtttgcttattggggccagggcacactggtcaccgtgtcctctgcttctaccaagggaccctctgtgttccctctggctccttccagcaagtctacctctggtggaaccgctgctctgggctgcctggtcaaggattactttcctgagcctgtgaccgtgtcttggaactctggtgctctgacctccggcgtgcacacatttccagctgtgctgcagtcctccggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtcctccatgtcctgctccagaactgctcggcggtccaagcgttttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacgcctccacctacagagtggtgtctgtgctgaccgtgctgcaccaggattggctgaacggcaaagagtacaagtgcgccgtgtccaacaaggccctgcctgctcctatcgaaaagaccatctccaaggctaagggccagcctcgggaacctcaggtttacacactgcctccatctcgggacgagctgaccaagaatcaggtgtccctgacctgcctcgtgaagggcttctacccttccgatatcgccgtggaatgggagtctaacggccagccagagaacaactacaagacaacccctcctgtgctggactctgacggctcattcttcctgtactccaagctgacagtggacaagtctcggtggcagcagggcaacgtgttctcctgttctgtgatgcacgaggccctgcacaaccactacacacagaagtctctgtctctgagccccggcaagtga

SEQ ID NO: 21 - DNA, 1347 bp, Artificial Sequence
  YB8 L2H5 L234A L235A K322A null Fc heavy chain
caggtgcagttggtgcagtctggcgccgaagtgaagaaacctggcgcttctgtgaaggtgtcctgcaaggcctctggctacacctttaccaactacgacatcaactgggtccgacaggctaccggacagggacttgagtggatgggatggattttccctggcgacggcagcacccagtacaacgagaagtttcagggcagagtgaccatgaccaccaacacctccatcagcaccgcctacatggaactgtccagcctgagatctgaggacaccgccgtgtactactgtgccagacagaccaccgccacttggtttgcttattggggccagggcacactggtcaccgtgtcctctgcttctaccaagggaccctctgtgttccctctggctccttccagcaagtctacctctggtggaaccgctgctctgggctgcctggtcaaggattactttcctgagcctgtgaccgtgtcttggaactctggtgctctgacctccggcgtgcacacatttccagctgtgctgcagtcctccggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtcctccatgtcctgctccagaagctgctggcggtccaagcgttttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcgccgtgtccaacaaggccctgcctgctcctatcgaaaagaccatctccaaggctaagggccagcctcgggaacctcaggtttacacactgcctccatctcgggacgagctgaccaagaatcaggtgtccctgacctgcctcgtgaagggcttctacccttccgatatcgccgtggaatgggagtctaacggccagccagagaacaactacaagacaacccctcctgtgctggactctgacggctcattcttcctgtactccaagctgacagtggacaagtctcggtggcagcagggcaacgtgttctcctgttctgtgatgcacgaggccctgcacaaccactacacacagaagtctctgtctctgagccccggcaagtga

SEQ ID NO: 22 - DNA, 1804 bp, Mus muscules
caggtgcagttggtgcagtctggcgccgaagtgaagaaacctggcgcttctgtgaaggtgtcctgcaaggcctctggctacacctttaccaactacgacatcaactgggtccgacaggccaccggacagtgtttggagtggatgggatggatcttccctggcgacggctctacccagtacaacgagaagtttcagggcagagtgaccatgaccaccaacacctccatcagcaccgcctacatggaactgtccagcctgagatctgaggacaccgccgtgtactactgtgccagacagaccaccgccacttggtttgcttattggggccagggcacactggtcacagtttctagcggaggcggaggaagtggtggcggaggttctggtggcggcggatcaggcggtggtggatctggcggcggtggaagtggcggaggcggctctgaaattgtgatgacccagtctcctgacttccagagcgtgacccctaaagagaaagtcaccatcacctgtcgggccagccagtccatctctgactacctgcactggtatcagcagaagcccgatcagtcccctaagctgctgattaagtacgccagccagagcatctccggcgtgccatccagattttctggctccggctctggctctgacttcaccctgaccatcaattccctggaagccgaggatgccgccacctactactgtcagaatggccacagcttccctctgaccttcggctgtggcaccaagctggaaatcaaaggcggcggaggctcaggcggaggtggaagcggaggtggcggttccggcggtggcggaagtcatgttcaactggttgaatccggcggaggattggtgcagccaggcggatctctgagactgtcttgtgccgcttccggcttctccctgactgattatggcgtgcactgggttcgacaagcccctggcaaaggactggaatggctgggagttatttggagcggcggaggaaccgcctacaacaccgctctgatctcccggttcaccatcagccgggacaactccaagaacaccctgtacctgcagatgaactctctgagagccgaagataccgctgtgtattactgcgctcggagaggcagctacccctacaactactttgatgcttggggctgcggaaccctggttacagtctcttctggcggcggaggcagcggaggtggtggttctggcggaggcggatctggtggcggaggtagtggcggcggtggaagcggtggcggaggatctcaagctgtggtcacacaagagcccagcctgacagtttctcctggcggaaccgttacactgacctgtggatcttctaccggcgctgtgaccgcctctaactacgctaactgggtgcagcagaaacccggccagtgtcctagaggcctgatcggcggacacaacaatagacctccaggcgtgcccgctagattctctggatctctgcttggcggcaaggctgctctgacacttttgggcgctcagcctgaggatgaggctgagtactattgcgccctgtggtactccgaccattgggttatcggcggagggaccaaactgaccgttctgggaacacctctgggcgacaccacacatacctctggaaagcctctggacggcgagtacttcacactgcagatccggggcagagaacgcttcgagatgttcagagagctgaacgaggccctggaactgaaggatgcccaggctggaaaagaacctggtggttcaggtggcgcccctcaccaccatcatcaccattaaggatccaagg

SEQ ID NO: 23 - protein, 442 aa, Mus muscules
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 24 - protein, 214 aa, Mus muscules
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 25 - protein, 5 aa, Mus muscules
NYDIN

SEQ ID NO: 26 - protein, 17 aa, Mus muscules
WIFPGDGSTQYNEKFKG

SEQ ID NO: 27 - protein, 9 aa, Mus muscules
QTTATWFAY

SEQ ID NO: 28 - protein, 11 aa, Mus muscules
RASQSISDYLH

SEQ ID NO: 29 - protein, 7 aa, Mus muscules
YASQSIS

SEQ ID NO: 30 - protein, 9 aa, Mus muscules
QNGHSFPLT

SEQ ID NO: 31 - protein, 534 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 32 - protein, 316 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 33 - protein, 4 aa, Homo sapiens
IRFD

SEQ ID NO: 34 - protein, 220 aa, Artificial Sequence
  Antibody ch8H9 Fab heavy chain
QVQLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKS

SEQ ID NO: 35 - protein, 213 aa, Artificial Sequence
  Antibody ch8H9 Fab light chain
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 36 - protein, 95 aa, Artificial Sequence
  IGKV3D-11
EIVLTQSPATLSLSPGERATLSCRASQSVSSYLAWYQQKPGQAPRLLIYDASNRATGIPARFSGSGPGTDFTLTISSLEPEDFAVYYCQQRSNWH

SEQ ID NO: 37 - protein, 95 aa, Artificial Sequence
  IGKV6-21
EIVLTQSPDFQSVTPKEKVTITCRASQSIGSSLHWYQQKPDQSPKLLIKYASQSFSGVPSRFSGSGSGTDFTLTINSLEAEDAATYYCHQSSSLP

SEQ ID NO: 38 - protein, 98 aa, Artificial Sequence
  IGHV1-8
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCAR

SEQ ID NO: 39 - protein, 98 aa, Artificial Sequence
  IGHV1-46
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAR

SEQ ID NO: 40 - protein, 23 aa, Artificial Sequence
  LFR1 VL L2H5
EIVMTQSPDFQSVTPKEKVTITC

SEQ ID NO: 41 - protein, 15 aa, Artificial Sequence
  LFR2 VL L2H5
WYQQKPDQSPKLLIK

SEQ ID NO: 42 - protein, 32 aa, Artificial Sequence
  LFR3 VL L2H5
GVPSRFSGSGSGSDFTLTINSLEAEDAATYYC

SEQ ID NO: 43 - protein, 10 aa, Artificial Sequence
  LFR4 VL L2H5
FGQGTKLEIK

SEQ ID NO: 44 - protein, 30 aa, Artificial Sequence
  HFR1 VH L2H5
QVQLVQSGAEVKKPGASVKVSCKASGYTFT

SEQ ID NO: 45 - protein, 14 aa, Artificial Sequence
  HFR2 VH L2H5
WVRQATGQGLEWMG

SEQ ID NO: 46 - protein, 32 aa, Artificial Sequence
  HFR3 VH L2H5
RVTMTTNTSISTAYMELSSLRSEDTAVYYCAR

SEQ ID NO: 47 - protein, 11 aa, Artificial Sequence
  HFR4 VH L2H5
WGQGTLVTVSS

SEQ ID NO: 48 - protein, 23 aa, Artificial Sequence
  LFR1 VL L2H3
EIVMTQSPDFQSVTPKEKVTITC

SEQ ID NO: 49 - protein, 15 aa, Artificial Sequence
  LFR2 VL L2H3
WYQQKPDQSPKLLIK

SEQ ID NO: 50 - protein, 32 aa, Artificial Sequence; identical to SEQ ID NO: 42
  LFR3 VL L2H3

SEQ ID NO: 51 - protein, 10 aa, Artificial Sequence
  LFR4 VL L2H3
FGQGTKLEIK

SEQ ID NO: 52 - protein, 30 aa, Artificial Sequence; identical to SEQ ID NO: 44
  HFR1 VH L2H3

SEQ ID NO: 53 - protein, 14 aa, Artificial Sequence
  HFR2 VH L2H3
WVRQATGQGLEWMG

SEQ ID NO: 54 - protein, 32 aa, Artificial Sequence
  HFR3 VH L2H3
RVTLTRNTSISTAYMELSSLRSEDTAVYFCAR

SEQ ID NO: 55 - protein, 11 aa, Artificial Sequence
  HFR4 VH L2H3
WGQGTLVTVSS

SEQ ID NO: 56 - protein, 23 aa, Artificial Sequence
  LFR1 VL L2H4
EIVMTQSPDFQSVTPKEKVTITC

SEQ ID NO: 57 - protein, 15 aa, Artificial Sequence
  LFR2 VL L2H4
WYQQKPDQSPKLLIK

SEQ ID NO: 58 - protein, 32 aa, Artificial Sequence; identical to SEQ ID NO: 42
  LFR3 VL L2H4

SEQ ID NO: 59 - protein, 10 aa, Artificial Sequence
  LFR4 VL L2H4
FGQGTKLEIK

SEQ ID NO: 60 - protein, 30 aa, Artificial Sequence; identical to SEQ ID NO: 44
  HFR1 VH L2H4

SEQ ID NO: 61 - protein, 14 aa, Artificial Sequence
  HFR2 VH L2H4
WVRQATGQGLEWIG

SEQ ID NO: 62 - protein, 32 aa, Artificial Sequence
  HFR3 VH L2H4
RVTMTTNTSISTAYMELSSLRSEDTAVYFCAR

SEQ ID NO: 63 - protein, 11 aa, Artificial Sequence
  HFR4 VH L2H4
WGQGTLVTVSS

SEQ ID NO: 64 - protein, 8 aa, Artificial Sequence
  IMGT CDR murine 8H9 heavy chain CDR1
GYTFTNYD

SEQ ID NO: 65 - protein, 8 aa, Artificial Sequence
  IMGT CDR murine 8H9 heavy chain CDR2
IFPGDGST

SEQ ID NO: 66 - protein, 11 aa, Artificial Sequence
  IMGT CDR murine 8H9 heavy chain CDR3
ARQTTATWFAY

SEQ ID NO: 67 - protein, 6 aa, Artificial Sequence
  IMGT CDR murine 8H9 light chain CDR1
QSISDY

SEQ ID NO: 68 - protein, 2 aa, Artificial Sequence
  IMGT CDR murine 8H9 light chain CDR2
YA

SEQ ID NO: 69 - protein, 9 aa, Artificial Sequence
  IMGT CDR murine 8H9 light chain CDR3
QNGHSFPLT

SEQ ID NO: 70 - protein, 8 aa, Artificial Sequence
  IMGT CDR H1 heavy chain CDR1
GYTFTNYD

SEQ ID NO: 71 - protein, 8 aa, Artificial Sequence
  IMGT CDR H1 heavy chain CDR2
IFPGDGST

SEQ ID NO: 72 - protein, 11 aa, Artificial Sequence
  IMGT CDR H1 heavy chain CDR3
ARQTTATWFAY

SEQ ID NO: 73 - protein, 6 aa, Artificial Sequence
  IMGT CDR L1 light chain CDR1
QSISDY

SEQ ID NO: 74 - protein, 2 aa, Artificial Sequence
  IMGT CDR L1 light chain CDR2
YA

SEQ ID NO: 75 - protein, 9 aa, Artificial Sequence
  IMGT CDR L1 light chain CDR3
QNGHSFPLT

SEQ ID NO: 76 - protein, 8 aa, Artificial Sequence
  IMGT CDR H2 heavy chain CDR1
GYTFTNYD

SEQ ID NO: 77 - protein, 8 aa, Artificial Sequence
  IMGT CDR H2 heavy chain CDR2
IFPGDGST

SEQ ID NO: 78 - protein, 11 aa, Artificial Sequence
  IMGT CDR H2 heavy chain CDR3
ARQTTATWFAY

SEQ ID NO: 79 - protein, 6 aa, Artificial Sequence
  IMGT CDR L2 light chain CDR1
QSISDY

SEQ ID NO: 80 - protein, 2 aa, Artificial Sequence
  IMGT CDR L2 light chain CDR2
YA

SEQ ID NO: 81 - protein, 9 aa, Artificial Sequence
  IMGT CDR L2 light chain CDR3
QNGHSFPLT

SEQ ID NO: 82 - protein, 8 aa, Artificial Sequence
  IMGT CDR H3 heavy chain CDR1
GYTFTNYD

SEQ ID NO: 83 - protein, 8 aa, Artificial Sequence
  IMGT CDR H3 heavy chain CDR2
IFPGDGST

SEQ ID NO: 84 - protein, 11 aa, Artificial Sequence
  IMGT CDR H3 heavy chain CDR3
ARQTTATWFAY

SEQ ID NO: 85 - protein, 6 aa, Artificial Sequence
  IMGT CDR L3 light chain CDR1
QSISDY

SEQ ID NO: 86 - protein, 2 aa, Artificial Sequence
  IMGT CDR L3 light chain CDR2
YA

SEQ ID NO: 87 - protein, 9 aa, Artificial Sequence
  IMGT CDR L3 light chain CDR3
QNGHSFPLT

SEQ ID NO: 88 - protein, 8 aa, Artificial Sequence
  IMGT CDR H4 heavy chain CDR1
GYTFTNYD

SEQ ID NO: 89 - protein, 8 aa, Artificial Sequence
  IMGT CDR H4 heavy chain CDR2
IFPGDGST

SEQ ID NO: 90 - protein, 11 aa, Artificial Sequence
  IMGT CDR H4 heavy chain CDR3
ARQTTATWFAY

SEQ ID NO: 91 - protein, 6 aa, Artificial Sequence
  IMGT CDR L4 light chain CDR1
QSISDY

SEQ ID NO: 92 - protein, 2 aa, Artificial Sequence
  IMGT CDR L4 light chain CDR2
YA

SEQ ID NO: 93 - protein, 9 aa, Artificial Sequence
  IMGT CDR L4 light chain CDR3
QNGHSFPLT

SEQ ID NO: 94 - protein, 8 aa, Artificial Sequence
  IMGT CDR H5 heavy chain CDR1
GYTFTNYD

SEQ ID NO: 95 - protein, 8 aa, Artificial Sequence
  IMGT CDR H5 heavy chain CDR2
IFPGDGST

SEQ ID NO: 96 - protein, 11 aa, Artificial Sequence
  IMGT CDR H5 heavy chain CDR3
ARQTTATWFAY

SEQ ID NO: 97 - protein, 8 aa, Artificial Sequence
  IMGT CDR H6 heavy chain CDR1
GYTFTNYD

SEQ ID NO: 98 - protein, 8 aa, Artificial Sequence
  IMGT CDR H6 heavy chain CDR2
IFPGDGST

SEQ ID NO: 99 - protein, 11 aa, Artificial Sequence
  IMGT CDR H6 heavy chain CDR3
ARQTTATWFAY

SEQ ID NO: 100 - protein, 23 aa, Mus muscules
DIVMTQSPATLSVTPGDRVSLSC

SEQ ID NO: 101 - protein, 15 aa, Mus muscules
WYQQKSHESPRLLIK

SEQ ID NO: 102 - protein, 32 aa, Mus muscules
GIPSRFSGSGSGSDFTLSINSVEPEDVGVYYC

SEQ ID NO: 103 - protein, 10 aa, Mus muscules
FGAGTKLELK

SEQ ID NO: 104 - protein, 30 aa, Mus muscules
QVQLQQSGAELVKPGASVKLSCKASGYTFT

SEQ ID NO: 105 - protein, 14 aa, Mus muscules
WVRQRPEQGLEWIG

SEQ ID NO: 106 - protein, 32 aa, Mus muscules
KATLTTDTSSSTAYMQLSRLTSEDSAVYFCAR

SEQ ID NO: 107 - protein, 11 aa, Mus muscules
WGQGTLVTVSA

SEQ ID NO: 108 - protein, 23 aa, Artificial Sequence
  L1 LFR1
EIVLTQSPDFQSVTPKEKVTLTC

SEQ ID NO: 109 - protein, 15 aa, Artificial Sequence
  L1 LFR2
WYQQKPDQSPKLLIK

SEQ ID NO: 110 - protein, 32 aa, Artificial Sequence; identical to SEQ ID NO: 42
  L1 LFR3

SEQ ID NO: 111 - protein, 10 aa, Artificial Sequence
  L1 LFR4
FGQGTKLEIK

SEQ ID NO: 112 - protein, 23 aa, Artificial Sequence
  L3 LFR1
EIVMTQSPDFQSVTPKEKVTLTC

SEQ ID NO: 113 - protein, 15 aa, Artificial Sequence
  L3 LFR2
WYQQKPDQSPKLLIK

SEQ ID NO: 114 - protein, 32 aa, Artificial Sequence
  L3 LFR3
GVPSRFSGSGSGSDFTLTINSLEAEDAGVYYC

SEQ ID NO: 115 - protein, 10 aa, Artificial Sequence
  L3 LFR4
FGQGTKLEIK

SEQ ID NO: 116 - protein, 23 aa, Artificial Sequence
  L4 LFR1
EIVMTQSPDFQSVTPKEKVTLTC

SEQ ID NO: 117 - protein, 15 aa, Artificial Sequence
  L4 LFR2
WYQQKPDQSPKLLIK

SEQ ID NO: 118 - protein, 32 aa, Artificial Sequence
  L4 LFR3
GIPSRFSGSGSGTDFTLTINSVEAEDAGVYYC

SEQ ID NO: 119 - protein, 10 aa, Artificial Sequence
  L4 LFR4
FGQGTKLEIK

SEQ ID NO: 120 - protein, 30 aa, Artificial Sequence; identical to SEQ ID NO: 44
  H1 HFR1

SEQ ID NO: 121 - protein, 14 aa, Artificial Sequence
  H1 HFR2
WVRQATGQGLEWIG

SEQ ID NO: 122 - protein, 32 aa, Artificial Sequence
  H1 HFR3
RVTLTTNTSISTAYMELSSLRSEDTAVYYCAR

SEQ ID NO: 123 - protein, 11 aa, Artificial Sequence
  H1 HFR4
WGQGTLVTVSS

SEQ ID NO: 124 - protein, 30 aa, Artificial Sequence
  H2 HFR1
QVQLQQSGAEVKKPGASVKVSCKASGYTFT

SEQ ID NO: 125 - protein, 14 aa, Artificial Sequence
  H2 HFR2
WVRQATGQGLEWIG

SEQ ID NO: 126 - protein, 32 aa, Artificial Sequence
  H2 HFR3
RATLTRNTSISTAYMELSSLRSEDTAVYYCAR

SEQ ID NO: 127 - protein, 11 aa, Artificial Sequence
  H2 HFR4
WGQGTLVTVSS

SEQ ID NO: 128 - protein, 30 aa, Artificial Sequence
  H6 HFR1
QVQLQQSGAEVKKPGASVKLSCKASGYTFT

SEQ ID NO: 129 - protein, 14 aa, Artificial Sequence
  H6 HFR2
WVRQATGQGLEWIG

SEQ ID NO: 130 - protein, 32 aa, Artificial Sequence
  H6 HFR3
RATLTTNTSISTAYMELSSLRSEDTAVYFCAR

SEQ ID NO: 131 - protein, 11 aa, Artificial Sequence
  H6 HFR4
WGQGTLVTVSS

SEQ ID NO: 132 - protein, 591 aa, Artificial Sequence
  3BH-3 without His tag
QVQLVQSGAEVKKPGASVKVSCKASGYTFTNYDINWVRQATGQCLEWMGWIFPGDGSTQYNEKFQGRVTMTTNTSISTAYMELSSLRSEDTAVYYCARQTTATWFAYWGQGTLVTVSSGGGGSGGGGSGGGGSGGGGSGGGGSGGGGSEIVMTQSPDFQSVTPKEKVTITCRASQSISDYLHWYQQKPDQSPKLLIKYASQSISGVPSRFSGSGSGSDFTLTINSLEAEDAATYYCQNGHSFPLTFGCGTKLEIKGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGAP

SEQ ID NO: 133 - protein, 10 aa, Artificial Sequence
  LFR4 VL L2H5 alternative
FGCGTKLEIK

SEQ ID NO: 134 - protein, 14 aa, Artificial Sequence
  HFR2 VH L2H5 alternative
WVRQATGQCLEWMG

SEQ ID NO: 135 - protein, 448 aa, Artificial Sequence
  YB8 H5 alternative
QVQLVQSGAEVKKPGASVKVSCKASGYTFTNYDINWVRQATGQCLEWMGWIFPGDGSTQYNEKFQGRVTMTTNTSISTAYMELSSLRSEDTAVYYCARQTTATWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 448 aa, Artificial Sequence
  YB8 H5 N297A and K322A alternative
QVQLVQSGAEVKKPGASVKVSCKASGYTFTNYDINWVRQATGQCLEWMGWIFPGDGSTQYNEKFQGRVTMTTNTSISTAYMELSSLRSEDTAVYYCARQTTATWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 448 aa, Artificial Sequence
  YB8_H5 L234A, L235A and K322A alternative
QVQLVQSGAEVKKPGASVKVSCKASGYTFTNYDINWVRQATGQCLEWMGWIFPGDGSTQYNEKFQGRVTMTTNTSISTAYMELSSLRSEDTAVYYCARQTTATWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 214 aa, Artificial Sequence
  YB8 L2 alternative
EIVMTQSPDFQSVTPKEKVTITCRASQSISDYLHWYQQKPDQSPKLLIKYASQSISGVPSRFSGSGSGSDFTLTINSLEAEDAATYYCQNGHSFPLTFGCGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - protein, 39 aa, Artificial Sequence
  Tetramerization domain, SADA polypeptide
KPLDGEYFTLQIRGRERFEMFRELNEALELKDAQAGKEP